(19) United States
(12) Patent Application Publication
(10) Pub. No.: US 2026/0081032 A1
(43) Pub. Date: Mar. 19, 2026

(54) Methods And Systems For Assessing Cumulative Ergonomic Risk

(71) Applicant: Dassault Systems Americas Corp., Waltham, MA (US)

(72) Inventors: Elham Ghorbani, Montreal (CA); Samira Keivanpour, Montreal (CA); Firdaous Sekkay, Blanville (CA); Daniel Imbeau, Montreal (CA); Julie Charland, Montreal (CA); David Brouillette, Saint-Lambert (CA)

(21) Appl. No.: 19/331,792

(22) Filed: Sep. 17, 2025

Related U.S. Application Data
(60) Provisional application No. 63/695,416, filed on Sep. 17, 2024.

(51) Int. Cl. G16H 50/30; G06F 30/12

(52) U.S. Cl. CPC G16H 50/30; G06F 30/12

(57) ABSTRACT

Embodiments assess cumulative ergonomic risk. An embodiment includes receiving risk data where the risk data may include, for each task of a plurality of tasks performed by an operator, an indication of an ergonomic risk level, of a plurality of ergonomic risk levels, for the operator performing the task. Such an embodiment continues by restructuring the risk data received to determine, across the plurality of tasks, a total time duration for each joint of a plurality of joints of the operator, at each risk level. Thereafter, the embodiment determines the cumulative ergonomic risk based on the total time duration for each joint at each risk level.

### RELATED APPLICATION

This application claims the benefit of U.S. Provisional Application No. 63/695,416, filed on Sep. 17, 2024. The entire teachings of the above application are incorporated herein by reference.

### BACKGROUND

A number of existing product and simulation systems are offered on the market for the design and simulation of objects, e.g., humans, parts, and assemblies of parts and actions, e.g., tasks and operations, amongst other examples. Such systems typically employ computer aided design (CAD) and/or computer aided engineering (CAE) programs. These systems allow a user to construct, manipulate, and simulate complex three-dimensional (3D) models of objects or assemblies of objects. These CAD and CAE systems, thus, provide a representation of modeled objects using edges, lines, faces, polygons, or closed volumes. Lines, edges, faces, polygons, and closed volumes may be represented in various manners, e.g., non-uniform rational basis-splines (NURBS).

CAD systems manage parts or assemblies of parts of modeled objects, which are mainly specifications of geometry. In particular, CAD files contain specifications, from which geometry is generated. From geometry, a representation is generated. Specifications, geometries, and representations may be stored in a single CAD file or multiple CAD files. CAD systems include graphic tools for representing the modeled objects to designers; these tools are dedicated to the display of complex objects. For example, an assembly may contain thousands of parts. A CAD system can be used to manage models of objects, which are stored in electronic files.

CAD and CAE systems use of a variety of CAD and CAE models to represent objects. These models may be programmed in such a way that the model has the properties (e.g., physical, material, or other physics based) of the underlying real-world object or objects that the model represents. Moreover, CAD/CAE models may be used to perform simulations of the real-word objects/environments that the models represent.

### SUMMARY

Simulating an operator, e.g., a human represented by a digital human model (DHM), in an environment is a common simulation task implemented and performed by CAD and CAE systems. Here, an operator refers to an entity which can observe and act upon an environment, e.g., a human, an animal, or a robot, amongst other examples. Computer-based operator simulations can be used to automatically predict behavior of an operator in an environment when performing a task with one or more objects. To illustrate one such example, these simulations can determine position and orientation of a human when assembling a car in a factory. The results of the simulations can, in turn, be used to improve the real-world physical environment. For example, simulation results may indicate that ergonomics or manufacturing efficiency can be improved by relocating objects in the environment.

Existing technologies offer functionality to evaluate risks for workers, e.g., before a production line is built or for purposes of improving an existing real-world workstation. However, evaluating risks using current DHM software requires ergonomics knowledge to interpret the results. Moreover, existing solutions for ergonomic analysis do not consider the cumulative burden of performing multiple tasks.

Embodiments solve these problems and provide improved functionality for evaluating risks, e.g., assessing ergonomic risks for workers.

One such embodiment is directed to a computer-implemented method of assessing cumulative ergonomic risk. Such an embodiment is implemented by a processor and includes, receiving, in memory of the processor, risk data. The risk data may include, for each task of a plurality of tasks performed by an operator, an indication of an ergonomic risk level, of a plurality of ergonomic risk levels, for the operator performing the task. The method continues by restructuring the risk data received to determine, across the plurality of tasks, a total time duration for each joint of a plurality of joints of the operator, at each risk level. Thereafter, the cumulative ergonomic risk is determined based on the total time duration for each joint at each risk level.

According to an embodiment, the indication of the ergonomic risk level for each task of the plurality of tasks includes a respective indication of ergonomic risk level for each joint of the plurality of joints of the operator performing the task.

An embodiment includes determining a cumulative ergonomic risk level for a subset of joints of the plurality of joints based on the total time duration for each joint of the subset at each risk level. In such an embodiment, the subset of joints may include (i) a right shoulder joint, a right elbow joint, and a right wrist joint, (ii) a left shoulder joint, a left elbow joint, and a left wrist joint, or (iii) neck joints and back joints.

In an embodiment, the cumulative ergonomic risk determined includes, for each joint of the plurality of joints, a respective indication of cumulative ergonomic risk across the plurality of tasks. In such an embodiment, each respective indication of cumulative ergonomic risk may be a given indication of ergonomic risk level from amongst the plurality of ergonomic risk levels. Further, in such an embodiment, determining the cumulative ergonomic risk may include for each joint of the plurality of joints, determining the respective indication of cumulative ergonomic risk across the plurality of tasks based upon (i) a comparison between the determined total time duration for the joint at a first risk level and a total time duration of the plurality of tasks and (ii) a comparison between the determined total time duration for the joint at a second risk level and the total time duration of the plurality of tasks. According to a further embodiment, the first risk level is a high-risk level and the second risk level is a medium risk level.

According to an embodiment, the plurality of tasks form an operation.

In yet another embodiment a first subset of the plurality of tasks form a first operation and a second subset of the plurality of tasks form a second operation. Further, in such an embodiment, determining the cumulative ergonomic risk based on the total time duration for each joint at each risk level may include identifying a cumulative ergonomic risk of the operator performing the first operation and identifying a cumulative ergonomic risk of the operator performing the second operation.

In an embodiment, at least one indication of ergonomic risk level is a function of operator posture and operator exerted force.

In another embodiment, the risk data received comprises data captured by a wearable device on an operator.

Embodiments may also include, responsive to the cumulative ergonomic risk exceeding a threshold, iteratively (i) determining modified risk levels for the operator performing each task of the plurality of tasks under modified operational conditions, (ii) restructuring the modified risk levels to determine, across the plurality of tasks, a modified total time duration for each joint, at each risk level, and (iii) determining modified cumulative ergonomic risk based on the modified total time duration for each joint at each risk level indicated, until the modified cumulative ergonomic risk is below the threshold. Such an embodiment may continue by modifying a real-world environment in accordance with the modified operational conditions for which the modified cumulative risk is below the threshold.

Another embodiment is directed toward a system for assessing cumulative ergonomic risk. According to an embodiment, the system includes a processor and a memory with computer code instructions stored thereon. In such an embodiment, the processor and the memory, with the computer code instructions, are configured to cause the system to implement any embodiments or combination of embodiments described herein.

Yet another embodiment is directed to a computer program product for assessing cumulative ergonomic risk. The computer program product comprises a non-transitory computer readable medium that includes program instructions which, when executed by a processor, causes the processor to implement any embodiments or combination of embodiments described herein.

It is noted that embodiments of the method, system, and computer program product may be configured to implement any embodiments, or combination of embodiments, described herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a flowchart of a method for assessing cumulative ergonomic risk, according to an embodiment.

FIGS. 1B and 1C illustrate functionality that may be implemented in the method of FIG. 1A.

FIG. 2 is a block diagram illustrating an example assembly line, with example workstations, that may be evaluated and modified using embodiments.

FIG. 3 is a flowchart of a workflow of a system for evaluating cumulative ergonomic risk according to an embodiment.

FIG. 4 is a diagram illustrating components of legacy Ergonomic Assessment Tools (EATs), their respective subcomponents, as well as embodiments of the present invention and their respective subcomponents that may be used to evaluate cumulative ergonomic risk.

FIG. 5 is a flow diagram illustrating a legacy Occupational Repetitive Action (OCRA) method and its subcomponents that may be analyzed and adapted for use in embodiments.

FIG. 6 is a diagram illustrating consideration of force by existing ergonomic assessment tools that may be used by embodiments.

FIG. 7 shows a decision tree of a method for identifying ergonomic risk according to an embodiment.

FIGS. 8A-8C illustrate elements of a workflow for determining cumulative ergonomic risk of an arm, according to an embodiment.

FIG. 9 illustrates a method for determining cumulative ergonomic risk of a trunk, according to an embodiment.

FIG. 10 illustrates a workflow for determining cumulative ergonomic risk of an upper limb, according to an embodiment.

FIG. 11 illustrates functionality for determining integrated ergonomic risk according to an embodiment.

FIG. 12 illustrates a comparison between risk assessment results determined using various methodologies.

FIG. 13 presents scenarios for evaluating ergonomic risk associated with the back, the shoulder, and the back and shoulder of an operator, as well as the scenarios' integrated ergonomic risks, according to an embodiment

FIGS. 14A-14C illustrate the application of embodiments to evaluate integrated ergonomic risk, for each group of scenarios illustrated in FIG. 13.

FIG. 15A is a chart illustrating a comparison of results of ergonomic evaluations performed using existing methods and embodiments, for the group of scenarios related to risk on the shoulder of FIG. 14A.

FIG. 15B is a chart illustrating a comparison of results of ergonomic evaluations performed using existing methods and embodiments, for the group of scenarios related to risk on the back of FIG. 14B.

FIG. 15C is a chart illustrating a comparison of results of ergonomic evaluations performed using existing methods and embodiments, for the group of scenarios related to risk on both shoulder and back of FIG. 14C.

FIG. 16 is a schematic view of a computer network in which embodiments may be implemented.

FIG. 17 is a block diagram illustrating an example embodiment of a computer node in the computer network of FIG. 16.

### DETAILED DESCRIPTION

A description of example embodiments follows.

Occupational ergonomics have a significant impact in the manufacturing world, from Musculoskeletal Disorders (MSD) to product quality issues. As such, assessing ergonomics using models, e.g., computer-based models, digital human models (DHMs), etc., is an important task for organizations, e.g., manufacturers.

Advantages of DHMs include the amount of biomechanical and anthropometrical data that is available. DHMs allow users to compare, model, simulate, optimize, and modify different scenarios, e.g., manufacturing environments and tasks, in a measurable way. Several applications, such as Santos, Jack (Siemens®), and DELMIA® Ergonomics (Dassault Systemes), allow the use of DHMs in a 3D manufacturing context.

As industries transition towards Industry 5.0, which emphasizes human-centric values and well-being, the importance of ergonomic risk assessment in workplace design has never been more critical. Existing ergonomic assessment tools have played a crucial role in evaluating and identifying potential risks; however, their limitations, particularly in virtual environments and digital human modeling systems, highlight the need for more comprehensive and adaptable methodologies. Embodiments (which may be referred to herein as Ergo4All-Pro™) solve the limitations of existing ergonomic assessment tools and provide a novel ergonomic risk assessment model designed to enhance existing virtual systems by providing a detailed evaluation of cumulative and integrated risks across various body parts.

Building upon the foundational Ergo4All™ model, embodiments incorporate insights from well-established methods such as Occupational repetitive Action (OCRA), Rapid Upper Limb Assessment (RULA), and Rapid Entire Body Assessment (REBA), while addressing challenges in assessing risks related to individual body parts, e.g., upper limb. Embodiments may leverage a fuzzy knowledge-based expert system to generate rules for predicting ergonomic risks, offering both categorical and score-based assessments. To validate their effectiveness, embodiments were applied to real-world industrial workstation and synthesized scenarios, with results compared to benchmark tools, including Ergonomic Assessment Worksheet (EAWS) and OCRA. The findings discussed herein demonstrate that embodiments not only align well with these benchmarks, but also provide a more refined assessment of ergonomic risks, particularly in areas that conventional tools may overlook. The ability of embodiments to evaluate cumulative risks in individual body parts can be used to optimize assembly lines (e.g., existing assembly lines and assembly lines being designed) and reduce subjective biases inherent in traditional assessments. Embodiments represent a significant advancement in ergonomic risk assessment, paving the way for safer, more efficient workplace designs in the era of Industry 5.0.

The increasing complexity of modern industrial environments, coupled with a growing emphasis on human-robot collaboration and automation, has amplified the importance of ergonomic risk assessment in workplace design. As industries progress towards Industry 5.0, which prioritizes human-centric values and well-being, there exists a need for advanced tools that can assess and mitigate ergonomic risks effectively. Traditional Ergonomic Assessment Tools (EATs) have been instrumental in evaluating specific body parts and identifying potential risks; however, their limitations, particularly in virtual environments and DHM systems, underscore the necessity for more comprehensive and adaptable methodologies.

The integration of virtual reality (VR) in ergonomic assessments facilitates a deeper understanding of spatial and environmental factors contributing to ergonomic risks, complementing the virtual design aspects of this research. The evolution of proactive ergonomic design approaches (Chaffin, 2005) and the importance of dynamic simulation in predicting and mitigating ergonomic risks (De Magistris et al., 2013) underscore the growing necessity of integrating human factors early in the design process (Da Silva et al., 2022) to ensure that ergonomic considerations are not an afterthought, but a fundamental aspect of design and assembly planning (Ahmed et al., 2021).

Ergonomic Workplace Design (EWD) is a tool developed by Dassault Systèmes, that helps engineers design safer and more efficient workplaces by applying DHM to avoid musculoskeletal disorders (MSDs) and their related expenses in the real world. (Dassault Systèmes, Ergonomic Workplace Design, 2024) EWD can apply an Ergo4All™ methodology that leverages existing standards to assess ergonomic risks in each body part for static tasks (U.S. Provisional Patent Application No. 63/287,251; U.S. patent application Ser. No. 18/063,338).

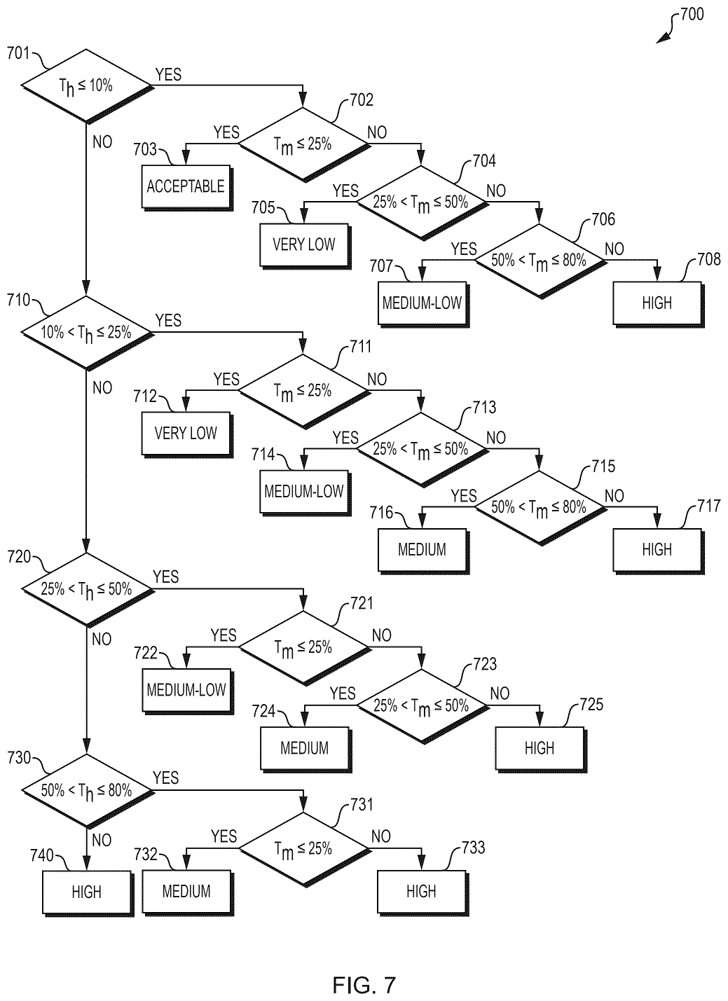

Embodiments seck to fulfil the industry need for a cumulative risk assessment tool and may build upon the foundation of existing ergonomics assessment tools, e.g., Ergo4All™. Embodiments provide a novel comprehensive approach that integrates time considerations into static ergonomic assessments, e.g., Ergo4All™. The integration of time considerations enables the evaluation of cumulative ergonomic risks across different body parts. An embodiment enhances existing ergonomic assessment methods by incorporating insights from the benchmark method, OCRA. Embodiments may employ a reverse engineering approach that identifies underlying logic behind time factor integration in OCRA. Further, embodiments may employ ergonomic knowledge-based expert systems and be implemented within EWD for dynamic ergonomic risk assessment. For example, these knowledge-based expert systems may be based on ergonomists' expertise and knowledge relating to details of ergonomic tools. (Ghorbani, 2024c). Said expertise has been applied to develop rules illustrated in, for example, TABLE IV and TABLE V, as well as method 700 of FIG. 7, discussed hereinbelow.

Embodiments may also evaluate cumulative risk in body part groupings, e.g., the upper limb. To achieve this, one such embodiment re-engineers the methodology and concepts behind RULA and REBA, and provides enhancements and advantages over RULA and REBA by implementing a customized approach for integrating risks across various body parts to generate a unique single score indicative of a cumulative ergonomic risk factor for a body part grouping, for example, the upper limb.

The novel evaluation functionality disclosed herein offers several key capabilities that enhance ergonomic assessments within DHM systems.

First, embodiments eliminate subjective effects of conventional EATs. Because embodiments may be based on biomechanical evaluation and perform assessments according to various standards such as EN1005-2, EN1005-3, EN1005-4, ISO 14738, ISO 11226, and ISO 11228-3 (Bourret et al., 2021), embodiments minimize subjective impacts found in traditional ergonomic checklists and tools.

Second, embodiments may evaluate each body part separately for workstations being evaluated, e.g., separately evaluating ergonomic risk for each body part at each workstation of a plurality of workstations. By detecting the cumulative risk level of each body part individually, this novel EAT enables ergonomic-oriented job rotation in Assembly Line Balancing Problems (ALBPs). Furthermore, embodiments assist decision makers in assigning collaborative robots (cobots) and supportive robots (Tong & Liu, 2021) based on identified risk points.

Third, embodiments can be used to optimize assembly/disassembly lines in the design phase. As a virtual assessment tool, embodiments enable the evaluation of workstations and entire lines based on various scenarios, allowing for optimization during the design phase to prevent future expenses related to corrective actions for ergonomic issues. Moreover, embodiments can also rely on measurements of existing real-world workstations/assembly lines to evaluate ergonomics of the real-world workstations/assembly lines. Results of these evaluations may, in turn, be used to modify and improve the existing workstation/assembly line, e.g., to reduce ergonomic risk and/or improve the manufacturing itself performed at the workstation/assembly line while not negatively impacting ergonomics.

Embodiments disclose a novel ergonomic risk assessment model designed to enhance existing DHM systems by providing a detailed evaluation of cumulative and integrated risks across various body parts. Embodiments can incorporate insights from well-established EATs such as OCRA, RULA, and REBA, while addressing gaps in traditional methods, particularly in assessing risks related to the neck and upper limbs. Embodiments may leverage a fuzzy knowledge-based expert system to generate rules for predicting ergonomic risks, offering both categorical and score-based assessments.

Discussed hereinbelow is a validation of the effectiveness and reliability of embodiments through application of embodiments to real-world industrial workstations and synthesized scenarios. By comparing the outputs from embodiments with those of benchmark EATs, the applicability of embodiments in both academic and industrial settings is demonstrated while also exploring the potential of embodiments to advance the field of ergonomic risk assessment.

The discussion below is structured as follows: a review of relevant literature is followed by an example methodology underlying the development of example embodiments. Thereafter, details of implementation of embodiments is discussed, followed by a discussion of results, including validation efforts and potential academic and industrial applications.

### Literature Review

The field of ergonomic risk analysis has significantly evolved with the integration of advanced technologies, including DHM, VR, and automation tools. The application of EATs within DHM systems for the design of assembly/disassembly workstations has become a growing area of interest, particularly in the contexts of Industry 4.0 and the emerging Industry 5.0. This trend reflects the need to mitigate the risks of Work-related Musculoskeletal Disorders (WMSDs) and enhance worker safety and productivity through innovative technologies. Recent studies have highlighted the evolution and application of various digital and virtual tools in ergonomic risk analysis, emphasizing the importance of precise posture analysis, real-time feedback, and the integration of human factors in the design process. Discussed hereinbelow is a synopsis of example relevant literature, focusing on key developments in ergonomic workstation design, the application of DHM systems, and novel methodologies that enhance the assessment of ergonomic risks.

### Digital Human Modeling & Ergonomic Assessment

DHM systems play a critical role in optimizing workstation design and reducing ergonomic risks through ergonomic assessment processes. Chaffin (2005) emphasized the proactive application of DHM tools in the design phase to pre-emptively address ergonomic concerns. Chaffin (2005) highlights the importance of integrating ergonomic principles early in the design process to minimize the risk of WMSDs by anticipating and mitigating potential issues before physical prototypes are developed. Further contributing to this concept, De Magistris et al. (2013) explored the dynamic control of DHM, enhancing the ability to conduct real-time ergonomic assessments. De Magistris et al. (2013) introduced innovative methods for simulating human motion with greater accuracy, which is crucial for identifying and correcting posture-related risks in dynamic work environments.

DHM systems offer substantial benefits by providing a virtual environment where human interactions with workstations, tools, and tasks can be simulated and analyzed without the need for physical prototypes. This enables detailed ergonomic analyses early in the design process, reducing the need for costly physical mock-ups and allowing for iterative testing and optimization. For instance, Paudel et al. (2022) introduced a 3D human pose estimation framework that utilizes video and image sequences to evaluate ergonomic postures in real-time. Paudel et al. (2022), which applies methods like Ovako Working Posture Analysis System (OWAS), REBA, and RULA, demonstrates high accuracy in scoring postural risks, underscoring the significance of precise posture analysis in preventing injuries in industrial settings.

Dahibhate et al. (2023) further explored the use of DHM systems in ergonomic design and product development, emphasizing the growing indispensability of DHMs across various industries. By simulating different body types and postures, DHMs enable designers to create products and work environments that serve a diverse workforce, enhancing both safety and comfort. This integration of ergonomic considerations from the outset contributes to the overall effectiveness of the product development process.

Understanding the capabilities and limitations of various DHM systems is critical for advancing ergonomic assessment tools. Poirson and Delangle (2013) conducted a comprehensive comparative analysis of different human modeling tools, providing valuable insights into the respective strengths and weaknesses of existing approaches. The analysis by Poirson and Delangle (2013) is particularly useful for researchers and practitioners seeking to select the most appropriate DHM tools for specific applications. Dahibhate et al. (2023) also highlighted how certain models are better suited for particular ergonomic assessments or industrial scenarios, guiding more informed decision-making in the adoption of digital human modeling technologies. Based on Poirson and Delangle (2013) and Dahibhate et al. (2023), the four most popular ergonomics-oriented DHM software are CATIA®-DELMIA®, Jack, RAMSIS, and AnyBody.

### Technological Innovations in Ergonomic Risk Assessment

Recent advancements in ergonomic risk assessment tools have seen a significant shift towards integrating digital technologies to enhance accuracy, efficiency, and user-friendliness. The integration of digital tools in ergonomic risk assessment has been significantly bolstered by advancements in VR and DHM technologies. Da Silva et al. (2022) conducted a comprehensive review of patents and literature, emphasizing how VR combined with DHM can improve ergonomic assessments during industrial product development. The findings by Da Silva et al. (2022) suggest that the fusion of these technologies provides a more immersive and accurate analysis of ergonomic risks, supporting a more effective design process aligned with Industry 4.0 and Industry 5.0 principles.

A notable innovation is the development of integrated solutions that combine wearable sensors with digital posture assessment methodologies, such as the time-based assessment computerized (TACOs) method. As detailed by Khamaisi et al. (2024), the TACOs approach allows for reliable postural assessments even by non-experts, accelerating analysis and providing enhanced qualitative data compared to traditional methods. The TACOs setup, which includes a wearable suit and proprietary software, has been tested in controlled industrial environments, demonstrating its potential to improve ergonomic evaluations in line with Industry 5.0 objectives.

Additionally, Emir et al. (2022) emphasized the significance of computer-assisted tools that specifically analyze working postures causing strain. Emir et al. (2022) focuses on the identification of high-risk postures through computational methods, emphasizing the need for ergonomic tools that can swiftly evaluate posture-related risks in various occupational settings.

### Virtual Reality & Automation in Ergonomic Design

The use of VR in ergonomic design has opened new avenues for real-time and immersive evaluation of postural risks. The potential of VR and DHM integration in ergonomic design is further exemplified by Da Silva et al. (2022), who highlighted how these technologies can bridge the gap between virtual simulations and real-world applications. By incorporating VR into DHM systems, designers can create more accurate simulations that enhance the identification and mitigation of ergonomic risks, leading to safer and more efficient work environments.

The ErgoVR tool, developed by Manghisi, et al. (2022), offers a VR-based approach to ergonomic design, allowing for both real-time and offline evaluation during the workstation design phase. This ErgoVR tool highlights the advantages of immersive, user-centered design processes in enhancing workstation ergonomics, providing designers with a more interactive and realistic platform for assessing ergonomic risks.

Moreover, the integration of automated design processes, as demonstrated by Beuss et al. (2023), shows promise in streamlining the ergonomic design of workstations through CAD models and human-in-the-loop decision-making. Beuss et al. (2023) integrates real-time human feedback within the design process, dynamically adjusting workstation parameters to ensure alignment with ergonomic standards and individual worker requirements.

### Application of DHMs in Assembly Process Planning

The integration of DHMs into assembly process planning and ergonomic design has gained significant attention, reflecting the growing need to optimize human-machine interactions and ensure worker safety and productivity. Ahmed et al. (2021) explored the benefits of integrating human factors early in the design process using DHM and surrogate modeling. Ahmed et al. (2021) demonstrates that early consideration of ergonomic factors through utilizing DHM significantly improves design outcomes, reducing the need for later-stage modifications and ensuring that ergonomic principles are embedded throughout the product lifecycle.

This approach aligns with Yin and Li's (2023) findings, which highlight the critical role of DHMs in simulating human tasks, evaluating ergonomic risks, and optimizing workstation layouts. Yin and Li (2023) underscores the versatility of DHMs in predicting potential ergonomic issues early in the design process, reducing the need for costly modifications during later stages of product development. Additionally, DHMs enable detailed analysis of human movements and postures, which is crucial for improving assembly efficiency and minimizing the risk of WMSDs.

### Contributions to the Literature

The literature reviewed above illustrates the significant progress in ergonomic risk analysis, particularly through the integration of digital technologies and human-centered design principles. The studies reviewed underscore the importance of developing tools that are not only technologically advanced, but also capable of providing holistic ergonomic assessments. However, the foregoing literature review has resulted in the identification of several key research gaps within the field of holistic ergonomic assessment method development. Embodiments contribute to this evolving field by introducing novel ergonomic assessment tools designed for use within DHM systems, addressing existing gaps and offering new possibilities for ergonomic workstation design. Thus, embodiments align with the broader industry movement towards more sophisticated, technology-driven ergonomic risk assessment functionality, which enhances both the precision and reliability of evaluations.

### Methodology

### Problem Description

Embodiments evaluate cumulative ergonomic risk in individual body parts as well as the integrated cumulative risk in groupings of body parts, e.g., upper limb, using a DHM system. Embodiments may leverage several standards and methods for assessing individual tasks.

The validation efforts discussed herein demonstrate that embodiments provide a reliable and valid tool for ergonomic risk assessment. The results from embodiments are consistent with established EATs, and show particular strength in evaluating risks that may be overlooked by other tools. These findings validate the capability of embodiments to provide accurate ergonomic assessments in both real-world and synthesized scenarios.

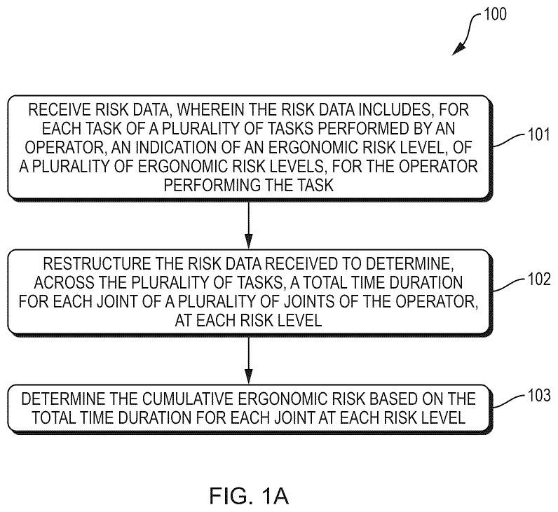

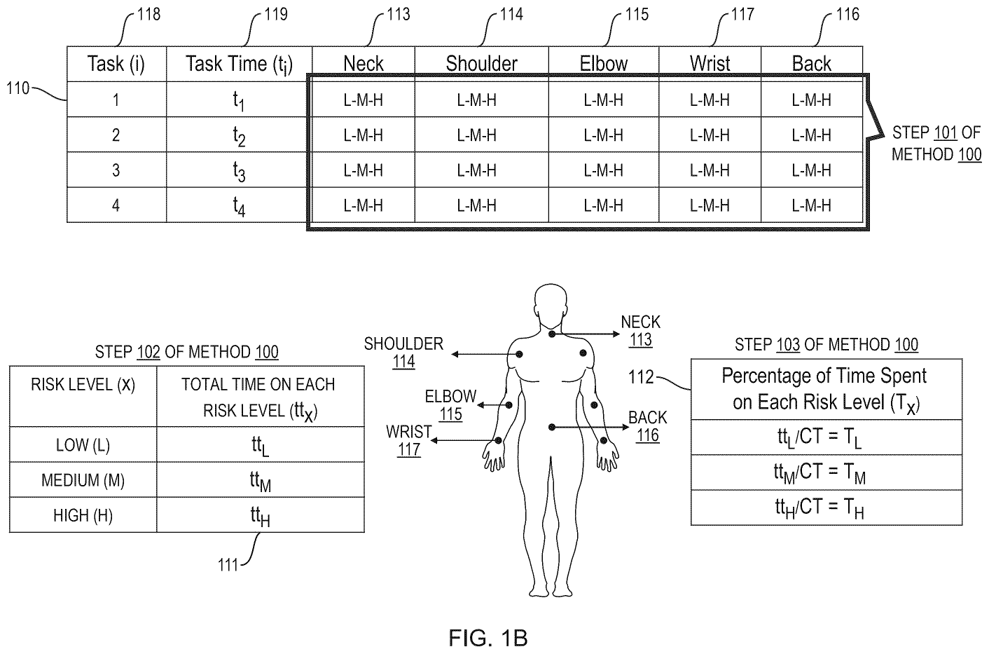

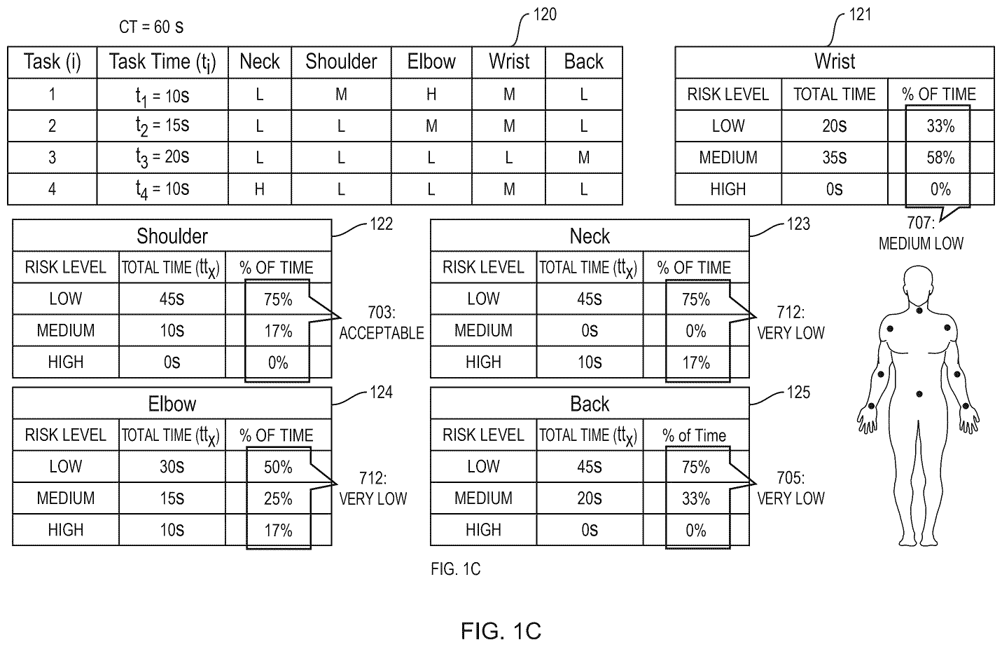

FIG. 1A is a flowchart of one such example embodiment. The method 100 begins at step 101 by receiving risk data. The risk data received at step 101 may include, for each task of a plurality of tasks performed by an operator (e.g., a human), an indication of an ergonomic risk level of a plurality of ergonomic risk levels, for the operator performing the task. Next, at step 102 the received risk data is restructured to determine, across the plurality of tasks, a total time duration for each joint of a plurality of joints of the operator, at each risk level. From there, at step 103, the method 100 determines the cumulative ergonomic risk based on the total time duration for each joint at each risk level. In an embodiment of the method 100 determining cumulative ergonomic risk at step 103 is determined in accordance with method 700 as discussed hereinbelow in relation to FIG. 7. Example details of steps 101-103 of method 100 are discussed hereinbelow at least in relation to FIGS. 1B and 1C.

The method 100 is computer-implemented and may be performed using any combination of hardware and software as is known in the art. For example, the method 100 may be implemented via one or more processors with associated memory storing computer code that causes the processor to implement steps 101-103 of the method 100.

Because the method 100 is computer implemented, the risk data may be received at step 101 from any location, memory, or data storage that can be communicatively coupled to a computing device implementing the method 100. Further, according to an embodiment, receiving the risk data at step 101 may include receiving a measurement from a sensor in a certain real-world environment in which tasks are performed. The sensor may be affixed to the operator and provide relevant gyroscopic or acceleration data, or may otherwise take measurements of the operator and/or environment. According to an embodiment, the risk rata received at step 101 may be from data captured by a wearable device on an operator.

The risk data received at step 101 can include any information or data known to those of skill in the art which indicates risk. Moreover, in an embodiment, indications of ergonomic risk received at step 101 may be a function of operator posture and operator exerted force. Moreover, receiving the indications of risk at step 101 may include determining (or receiving indications previously determined) through static evaluation performed using one or more models and ISO standards. For example, standards such as EN1005-2, EN1005-3, EN 1005-4, ISO 14738, ISO 11226, ISO 11228-3 and models such as existing 3D static biomechanical models (e.g., Ergo4All™) that may include a simulated manakin at a workstation as a representation of operator posture. Through static evaluation-joint load, joint angle, hand position, and object weight may be considered to assess risk level in each joint for performing each task. Further, the risk data received at step 101 may be output from one or more existing EAT. For instance, an embodiment may obtain risk data from Ergo4All™ where Ergo4All™ was used to evaluate the ergonomic risk of each task individually. Further still, receiving the risk data at step 101 may include implementing an EAT that individually assesses ergonomic risk for each task.

According to an embodiment of the method 100, the indication of ergonomic risk level for each task of the plurality of tasks received at step 101 may include a respective indication of ergonomic risk level (e.g., in the form of an indication of time duration based on time of executing each task) for each joint (e.g., wrist, elbow, shoulder) of the plurality of joints of the operator performing the task. Thus, in such an embodiment, the risk data received at step 101 includes, for each task of a plurality of tasks performed by an operator (e.g., a human), an indication of an ergonomic risk level of a plurality of ergonomic risk levels, for each joint of the operator performing the task. To illustrate, consider a simplified example where an operator has an elbow and shoulder joint and performs two tasks, A and B. In this illustrative example, ergonomics of the operator performing tasks A and B individually is performed using a method that output an indication of ergonomic risk as being low, medium, or high. Thus, in such an example embodiment, the data received at step 101 may indicate that for task A, elbow ergonomic risk is medium and shoulder ergonomic risk is high; while for task B, elbow ergonomic risk is high and shoulder ergonomic risk is high.

An embodiment of the method 100 further includes determining a cumulative ergonomic risk level for a subset of joints of the plurality of joints based on the total time duration for each joint of the subset at each risk level (See FIG. 1B, FIG. 1C, and FIG. 7 discussed hereinbelow). In such an embodiment, the subset of joints may include (i) a right shoulder joint, a right elbow joint, and a right wrist joint, (ii) a left shoulder joint, a left elbow joint, and a left wrist joint, or (iii) neck joints and back joints.

Embodiments determine cumulative ergonomic risk. To illustrate, posture and applied force may determine the loading at a joint (e.g., lifting an object with a hand from the ground to a shelf at 6 feet above the ground). A risk level (e.g., risk data) may be associated with the posture at the time the load is about to be released by the operator and placed on the shelf. It is at this moment when the shoulder is maximally loaded (i.e., just before the release of the object). If this task of lifting the object to the shelf is repeated, then shoulder fatigue may become a risk depending on (i) how many times during a given work period the task is repeated and (ii) the weight of the load. Therefore, according to an embodiment, “cumulative” may be understood to be associated with the repetitive loading of the shoulder (and/or any joint or grouping of joints during a respective task) over a time duration. As such, the risk of shoulder fatigue over the task duration may be associated with an ergonomic risk referred to as “cumulative” because embodiments consider several repetitions of the same or similar loading situations for a given joint. Further, embodiments may also determine the cumulative risk to any joint(s) or grouping of joint(s) over performing multiple, e.g., unique, tasks.

In an embodiment of the method 100, the cumulative ergonomic risk determined at step 103 may include, for each joint of the plurality of joints, a respective indication of cumulative ergonomic risk across the plurality of tasks. In such an embodiment, each respective indication of cumulative ergonomic risk may be a given indication of ergonomic risk level from amongst the plurality of ergonomic risk levels. (See FIG. 1B, FIG. 1C, and FIG. 7 discussed hereinbelow). For instance, in the example of lifting an object with a hand from the ground to a shelf described above, each lift of a 2 kg load may be associated with a risk level (e.g., risk data). A second risk level may be associated with the same lift, but with a lighter 1 kg load, and a third risk level may be associated with the same lift, but for a 5 kg load. The cumulative ergonomic risk level would be a risk level that combines the three risks associated with each respective load for an overall risk level for the joint, in this instance the shoulder. This risk level may take into account the number of lifts performed with each load, over a duration. For example, 30 lifts performed during a 30-min work duration, that is, 10 lifts with each load (10 lifts with the 2 kg load, 10 lifts with the 1 kg load, and 10 lifts with the 5 kg load). The resulting cumulative ergonomic risk level would combine the three risk levels (one for each load) and also consider frequency or occurrence for each during the work duration.

Further, in an embodiment of the method 100, determining the cumulative ergonomic risk may further include, for each joint of the plurality of joints, determining the respective indication of cumulative ergonomic risk across the plurality of tasks based upon (i) a comparison between the determined total time duration for the joint at a first risk level and a total time duration of the plurality of tasks and (ii) a comparison between the determined total time duration for the joint at a second risk level and the total time duration of the plurality of tasks. For example, according to an embodiment, the first risk level may be a “high” risk level, and the second risk level may be a “medium” risk level. (See FIG. 1B, FIG. 1C, and FIG. 7 discussed hereinbelow). For instance, returning to the shelf example, an embodiment may compare the total time duration for the 5 kg lift and its associated risk level (e.g., a high risk level) with the total time duration for the plurality of tasks, and also compare the total time duration for the 2 kg lift and its associated risk level (e.g., a medium risk level) with the total time duration for the plurality of tasks. Based on the respective amount of time spent lifting the 5 kg load, an embodiment may determine a first risk level (e.g., a high risk level); and based on the respective amount of time spent lifting the 2 kg load, an embodiment may determine a second risk level (e.g., a medium risk level). Based on the determined first risk level (high risk) and the determined second risk level (medium risk), an embodiment may determine cumulative ergonomic risk across these plurality of tasks.

In an embodiment, the plurality of tasks form an operation. For example, an operation may be understood as a sequence of task elements or tasks. For instance, returning to the shelf example, putting the object on the shelf may be considered an operation. This operation may involve the following tasks: (1) reaching for the part on the cart, (2) grasping the part with the right hand, (3) moving the part at reading distance from eye, (4) turning the part to see the part's bottom for reading a part number, (5) moving the part to the corresponding address number on the shelf's front, (6) releasing the part on the shelf, (7) moving the arm back to neutral position.

According to another embodiment, a first subset of the plurality of tasks form a first operation and a second subset of the plurality of tasks form a second operation. In such an embodiment, determining the cumulative ergonomic risk based on the total time duration for each joint at each risk level at step 103 includes identifying a cumulative ergonomic risk of the operator performing the first operation and identifying a cumulative ergonomic risk of the operator performing the second operation. (See FIG. 1B, FIG. 1C, and FIG. 7 discussed hereinbelow). For instance, in the shelf example described above, a subset of the identified tasks (e.g., tasks 1-4) may be considered a first operation and another subset of the identified tasks (e.g., tasks 5-7) may be considered a second operation. In such an example embodiment, a cumulative risk of the first operation (performing tasks 1-4 may be determined) and a cumulative risk of the second operation (tasks 5-7) may be determined. In such an embodiment, the data pertaining to each grouping of tasks (1-4 and 5-7) are used as described herein to determine the cumulative risk of the first operation and the second operation.

An embodiment of the method 100 may further include, responsive to the cumulative ergonomic risk exceeding a threshold, iteratively (i) determining modified risk levels for the operator performing each task of the plurality of tasks under modified operational conditions, (ii) restructuring the modified risk levels to determine, across the plurality of tasks, a modified total time duration for each joint, at each risk level, and (iii) determining modified cumulative ergonomic risk based on the modified total time duration for each joint at each risk level indicated, until the modified cumulative ergonomic risk is below the threshold. Such an embodiment may further include modifying a real-world environment in accordance with the modified operational conditions for which the modified cumulative risk is below the threshold. To illustrate, if the risk level for a wrist joint performing multiple tasks is determined to be “very high,” an embodiment may modify operating conditions of one or more of the multiple tasks and reevaluate the cumulative risk across the multiple task. For instance, if one of the original tasks includes a wrist rotation, this wrist rotation may be eliminated (i.e., modifying the operational conditions). Thereafter, such an embodiment may restructure the risk data (which includes risk data for a task that no longer includes the wrist rotation) to determine a modified total time duration for each joint (e.g., the wrist) at each risk level and, in turn, determine a modified cumulative ergonomic risk for each joint (including the wrist). Assuming all other tasks are the same, the modified risk level for the wrist should be lower than the unmodified risk level. This process of determining the modified cumulative ergonomic risk based on the modified total time duration for each joint (i.e., the wrist) at each risk level, may be repeated until the modified cumulative ergonomic risk is below a desired level, i.e., threshold.

Embodiments of the method 100 may be utilized to assess a real-world environments, e.g., a workstation at a factory, and results can be utilized to modify the real-world environment, e.g., to improve ergonomics. From the example above of placing an object on a shelf, embodiments may determine that having the part number printed on the bottom of the part causes negative ergonomic impact to, for example, the wrist, and, responsively, such an embodiment may instead print the part number on the top. Thereby eliminating task (4) and reducing the risk to the wrist. Further, embodiments may indicate that lifting a heavy object causes significant risk and, thus, the lifting of the object may be performed using a machine to reduce risk to the shoulder.

As described herein, embodiments, e.g., the method 100, may utilize existing EATs such as Ergo4All™ and OCRA to individually assess ergonomics of tasks, such as at step 101. It is noted that the definition of “task” in EATs is not uniform. For instance, the definition of task in Ergo4All™ differs from other EATs like OCRA. Thus, when utilizing existing EATs, embodiments may account for different definitions of tasks. According to an embodiment, tasks are defined as the smallest units of activity requiring force, taking a specific amount of time, and identifiable based on a defined posture.

FIG. 1B illustrates example details for steps 101, 102, and 103 of the method 100 from FIG. 1A, according to an embodiment. Table 110 in FIG. 1B illustrates example risk data that may be received at step 101 of method 100, according to an embodiment. In an embodiment, data received at step 101 may include an indication of risk levels (for example, from Ergo4All™, See U.S. Provisional Patent Application No. 63/287,251 and U.S. patent application Ser. No. 18/063,338) for the neck 113, shoulder 114, elbow 115, wrist 117, and back 116, for each task 118 (with an associated task time 119). Such an indication of risk level received at step 101 of the method 100 as illustrated by table 110 may be one or “L” for low risk, “M” for medium risk, or “H” for high risk.

Table 111 in FIG. 1B illustrates example details for step 102 of method 100, namely, for each joint, calculating the summation of time that an operator spends on performing tasks in each risk level (i.e., restructuring the risk data received to determine, across the plurality of tasks, a total time duration for each joint of a plurality of joints of the operator, at each risk level). As indicated in Table 111 represented in FIG. 1B, “Risk Level (x)” may be “Low (L),” “Medium (M),” or “High (H).” Each “Risk Level (X)” from Table 111 has an associated “Total Time on Each Risk Level (ttₓ), where ttₓ is the summation of task duration in risk level “x” (i.e., low (L), medium (M), or high (H)). In an embodiment, at step 102, the table 111 is determined for each joint. In other words, in such an embodiment, tt_{L}. (total time at low risk), tt_{M} (total time at medium risk), and tt_{H} (total time at high risk) is determined at step 102 for each joint. For the risk level “Low (L),” the corresponding Total Time may be written as (tt_{L}), for the risk level “Medium (M),” the corresponding Total Time may be written as (tt_{H}), and for the risk level “High (H),” the corresponding Total Time may be written as (tt_{H}). For example, once an indication of risk level is received at step 101, for example, high risk (H) for the neck 113 during Task 4, step 102 restructures the received risk data (i.e., by identifying which task of the plurality of tasks corresponds to a respective risk level, as well as each task's duration) to determine a total time duration (ttₓ) the neck 113 is at the high risk (H) level (i.e., (tt_{H})).

Table 112 in FIG. 1B illustrates further details for step 103 of method 100. Namely, for each joint, the method 100, at step 103, may evaluate the percentage of time (Tx) that an operator spends on performing tasks in each risk level by dividing the total time spent at each risk level by the CT, to assess the cumulative risk on that joint. In this way, such an embodiment determines a table 112 for each joint at step 103. In an embodiment, tables 112 may be determined for each joint using tables 111 for each joint and the CT. For example, T_{L} (percentage of time spent in a low risk level) may be calculated as T_{L}=tt_{L}/CT, T_{M} (percentage of time spent in a medium risk level) may be calculated as T_{M}=ttₘ. CT, and T_{H} (percentage of time spent in a high risk level) may be calculated as T_{H}=tt_{H}/CT. This process may be further detailed in relation to method 700 of FIG. 7, discussed hereinbelow.

FIG. 1C illustrates further details of steps 101-103 of the method 100 by providing an example comprising four tasks (numbered 1-4) where each of the tasks has a different duration and different associated risk level on each body part. The CT for each of these four tasks is 60 seconds. Table 120 of FIG. 1C is an example of risk data that may be received at step 101 of the method 100. The table 120 includes the example tasks 1-4 and, for each task (i) associated task times (tᵢ) and the risk levels associated with the neck, shoulder, elbow, wrist and back, for each task (i). Tables 121-125 of FIG. 1C illustrate the calculated percentage of time each joint (i.e., wrist, shoulder, neck, elbow and back, respectively) spends in each risk level.

To illustrate, consider the example of the wrist. Table 120 (e.g., received at step 101) indicates that the wrist was in medium risk for t₁ (10 s), t₂ 15 s, and t₄ (10 s) and the risk was in low risk for 20 s. At step 102, this data would be restructured (as shown in table 121) to indicate that the wrist was in low risk for 20 s, medium risk for 35 s, and high risk for 0 s. This restructured data is then used with a CT of 60 s (e.g., at step 103) to determine the percentage of time for the wrist at each risk level (i.e., the cumulative ergonomic risk based on the total time duration). For the wrist, as shown by table 121 this results in a total percentage of time spent in of 33% (low), 58% (medium), and 0% (high). This corresponds to a risk level of “medium low,” which may be determined, e.g., as part of the method 100, using the fuzzy rules represented in the method 700 of FIG. 7 discussed hereinbelow. To continue, the foregoing steps are performed for each joint. Thus, table 122 illustrates that for the CT, the shoulder was in a low risk level for 45 seconds, a medium risk level for 10 seconds, and a high risk level for 0 seconds, corresponding to a total percentage of time spent in each risk level of 75%, 17%, and 0%, respectively. This corresponds to a risk level of “Acceptable,” which may be determined by the fuzzy rules represented in the method 700 of FIG. 7 discussed hereinbelow. Further still, table 123 illustrates that for the CT, the neck was in a low risk level for 45 seconds, a medium risk level for 0 seconds, and a high risk level for 10 seconds, corresponding to a total percentage of time spent in each risk level of 75%, 0%, and 17%, respectively. This corresponds to a risk level of “Very Low,” which may be determined by the fuzzy rules represented in the method 700 of FIG. 7 discussed hereinbelow. Table 124 illustrates that for the CT, the elbow was in a low risk level for 30 seconds, a medium risk level for 15 seconds, and a high risk level for 10 seconds, corresponding to a total percentage of time spent in each risk level of 50%, 25%, and 17%, respectively. This corresponds to a risk level of “Very Low,” which may be determined by the fuzzy rules represented in the method 700 of FIG. 7 discussed hereinbelow. In addition, table 125 illustrates that for the CT, the back was in a low risk level for 45 seconds, a medium risk level for 20 seconds, and a high risk level for 0 seconds, corresponding to a total percentage of time spent in each risk level of 75%, 33%, and 0%, respectively. This corresponds to a risk level of “Very Low,” which may be determined by the fuzzy rules represented in the method 700 of FIG. 7 discussed hereinbelow.

In the example presented in FIG. 1C, among all the joints (wrist, shoulder, neck, elbow, and back) the wrist was considered to be of the highest ergonomic risk. According to an embodiment, a meaningful change in the position of the object or the angle of the wrist joint may reduce the “medium risk” task to “low risk” and thereby mitigate the cumulative ergonomic risk associated with the wrist. Such an embodiment may be said to assess a real-world environment, and modify the real-world environment in order to improve ergonomics.

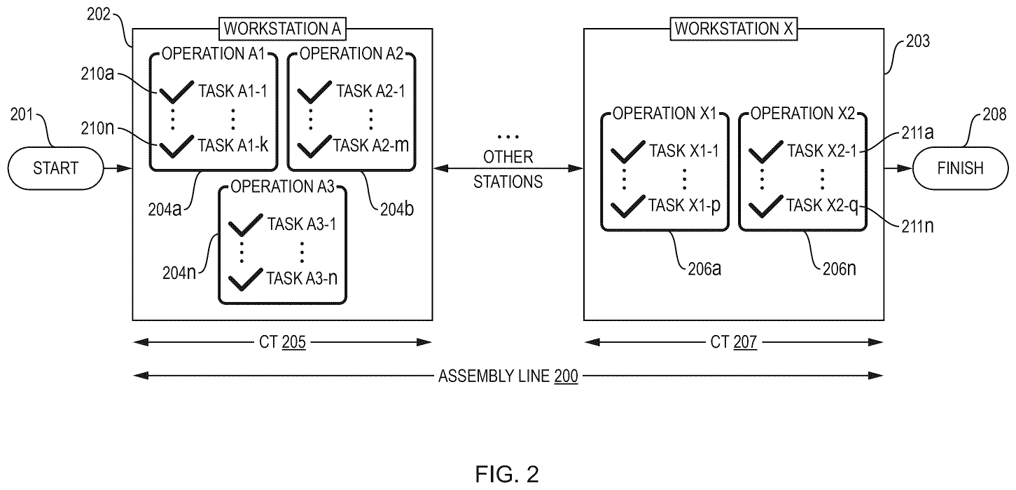

As shown in FIG. 2, discussed hereinbelow, each workstation in an assembly line may contain several tasks, which can be categorized into operations based on their purpose and goal.

FIG. 2 illustrates an example assembly line 200 for which embodiments, e.g., method 100, may be used to evaluate cumulative ergonomic risk, e.g., risk across the entire assembly line 200. The example assembly line 200 includes workstations 202 and 203 and starts 201 and continues from workstation A 202 through any number of workstations before workstation X 203 and finishing 208 Workstation A 202, defined by cycle time (CT) 205 (e.g., the actual amount of time it takes to complete one specific task or unit of a process, from start to finish, including all active work, waiting, and manual effort), includes three operations 204a-n, where each operation 204a-n each includes a plurality of tasks, e.g., 210a-n. Workstation X, defined by CT 207, contains two operations 206a-n, each composed of a plurality of tasks, e.g., 211a-n. A workstation, e.g., 202, may contain any number of operations, e.g., 204a-n, and an operation may contain any number of tasks, e.g., 210a-n.

While existing EATs, e.g., Ergo4All™, can evaluate ergonomic risk of tasks individually, existing EATs cannot evaluate the cumulative risk of performing multiple tasks. In contrast, embodiments assess cumulative risk of performing multiple tasks. For instance, embodiments can determine the cumulative risk of performing tasks at workstation 202 (FIG. 2). Embodiments can also determine cumulative ergonomic risk across any number of workstations, e.g., workstations 202-203 (FIG. 2). Embodiments can enhance existing EAT methods by incorporating insights from benchmark methods, including OCRA, RULA and REBA.

An initial stage of developing an embodiment involved a detailed study of OCRA to identify key sections that incorporate time factors, enabling the development of an assessment tool capable of evaluating cumulative ergonomic risk in each body part. Subsequently, based on RULA and REBA methodologies, an embodiment provides a method for evaluating the cumulative integrated ergonomic risk of, for example, the upper limb.

### Proposed Framework

Ergo4All™ is a static tool based on several standards including EN1005-2, EN1005-3, EN1005-4, ISO 14738, ISO 11226, and ISO 11228-3, to assess ergonomic risk levels in each joint for one task (Bourret et al., 2021). Embodiments disclosed herein provide enhancements for existing EATs and provide a comprehensive dynamic tool that evaluates not only the cumulative risk in each body joint, but also the total risk in grouping(s) of joint, e.g., upper limb.

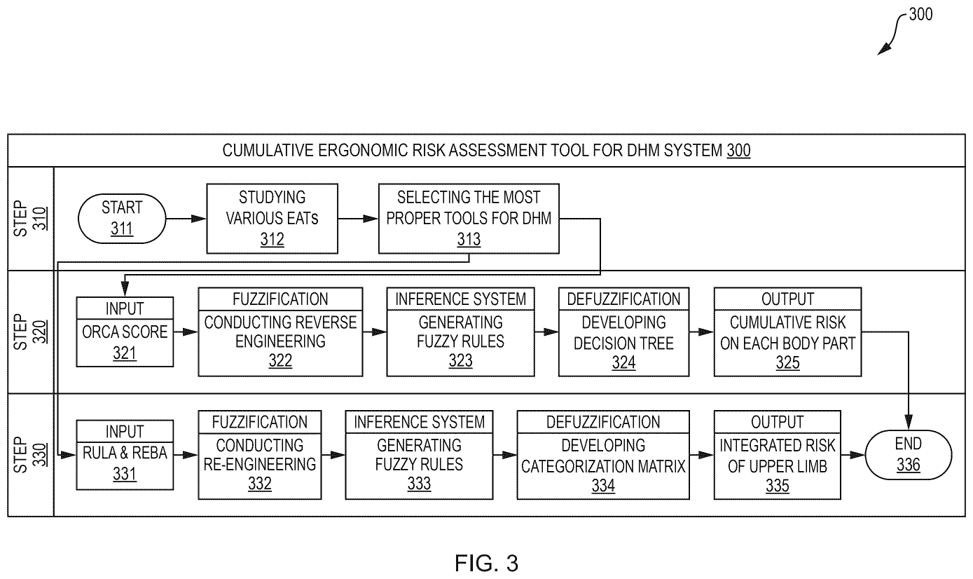

FIG. 3 is a flowchart of a workflow 300 for evaluating cumulative ergonomic risk according to an embodiment. According to an embodiment, workflow 300 determines cumulative ergonomic risk for DHM systems. Workflow 300 includes step 310, step 320, and step 330. Beginning with step 310, the workflow 300 starts at step 311 and then studies various EATs (e.g., EAWS, OCRA, Borg Scales, RULA, REBA, etc.) at step 312. Thereafter, at step 313, the workflow 300 selects the most proper tools for DHM. Selecting the most proper tool at step 313 may include, for example, comparing the characteristics of each method including input, output, weakness, strengths, and requirements. From there, the workflow 300 may either go to step 320 or 330, depending upon the existing EAT selected at step 313.

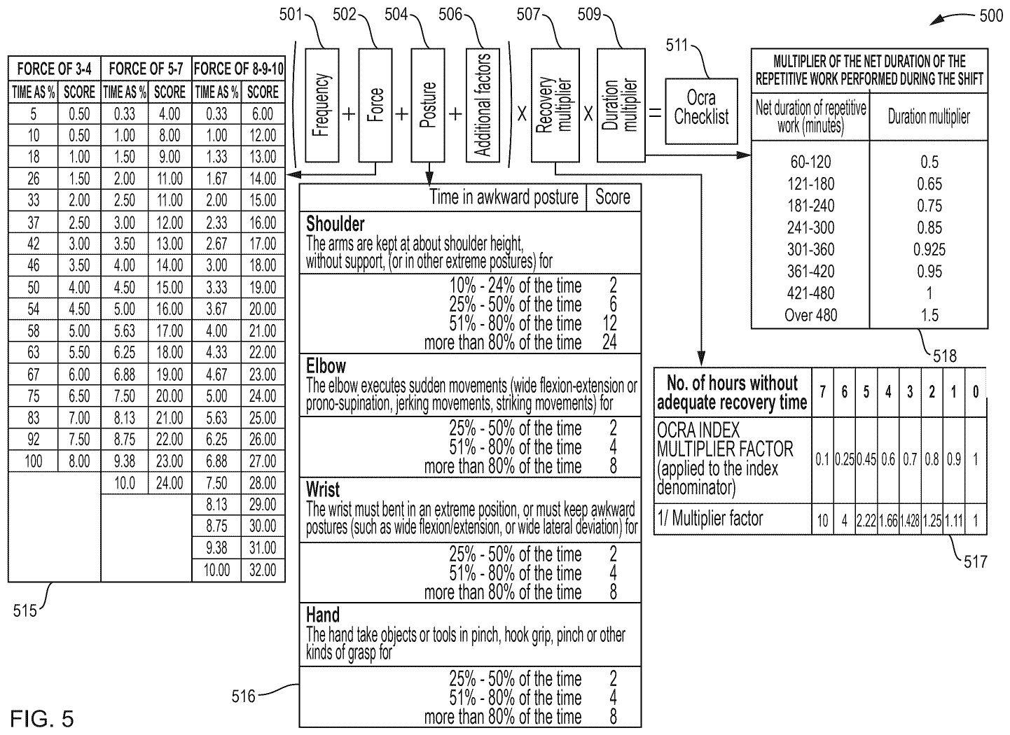

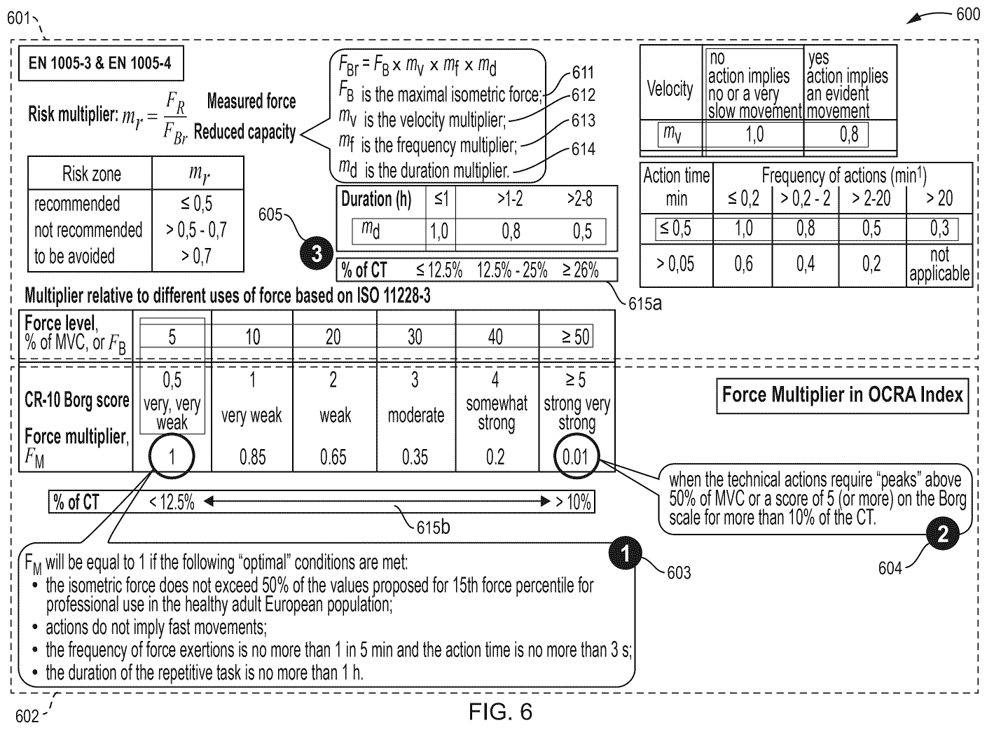

If OCRA is selected at step 313, the method 300 moves to step 320 and an OCRA score is input/received at step 321. To continue step 320, after the OCRA score is input at step 321, the workflow 300 performs fuzzification 322 by conducting reverse engineering. For example, as discussed hereinbelow at least in relation to FIG. 5, OCRA was analyzed (by reverse engineering) by starting from OCRA's output. Each step and concept of OCRA, from the output to the input, was analyzed for subjective concepts (i.e., fuzzy logic) in order to simplify the method for regenerating OCRA in a DHM environment. Thereafter, at step 323 an inference system is used to generate fuzzy rules, and defuzzification at step 324 develops a decision tree. For example, as discussed hereinbelow at least in relation to both FIG. 6 and FIG. 7, knowledge from ergonomic experts as well as portions of relevant standards (e.g., EN 1005-3 and EN 1005-4) were comparted to the OCRA method, and the findings of said comparison were used to shape the inference system utilized at step 323; and the decision tree developed at step 324 (See FIG. 7) illustrates the de-fuzzified results of the developed inference system.) The method 300 then moves to step 325 where a cumulative risk on each body part is output. The method 300 may then move to end 336, or move to generating fuzzy rules 333 of step 330.

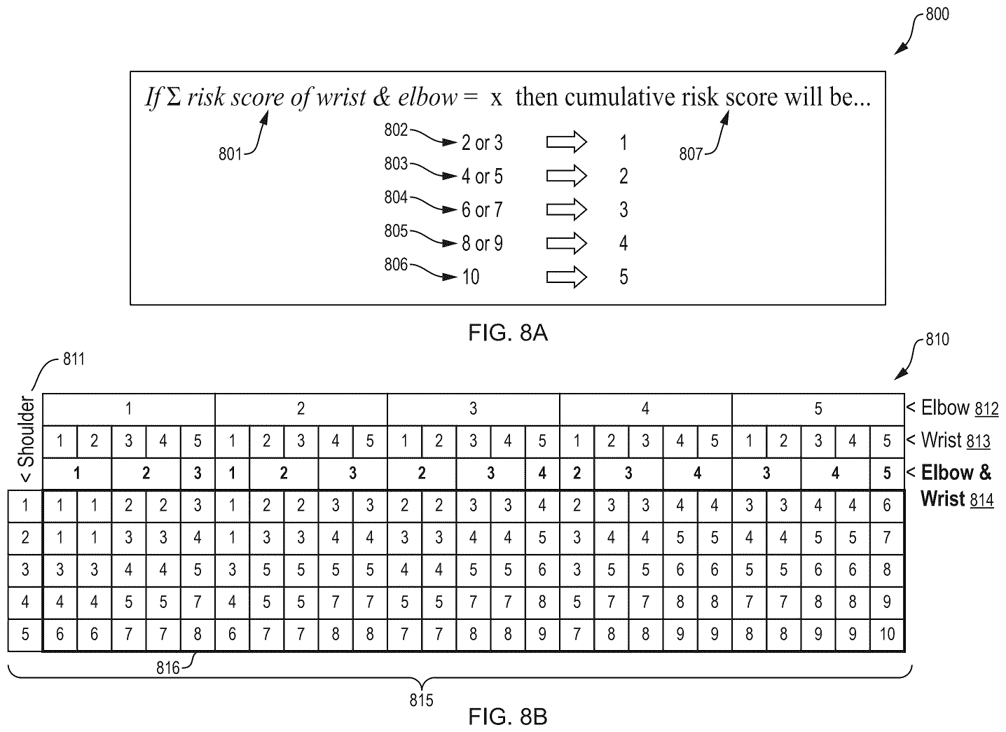

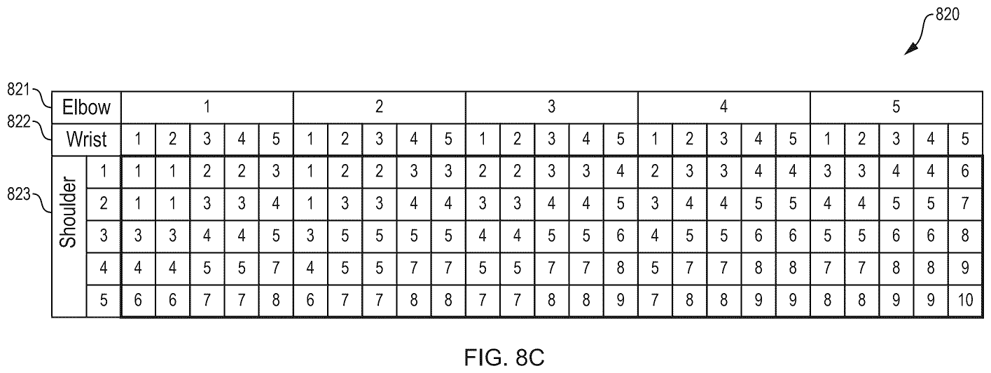

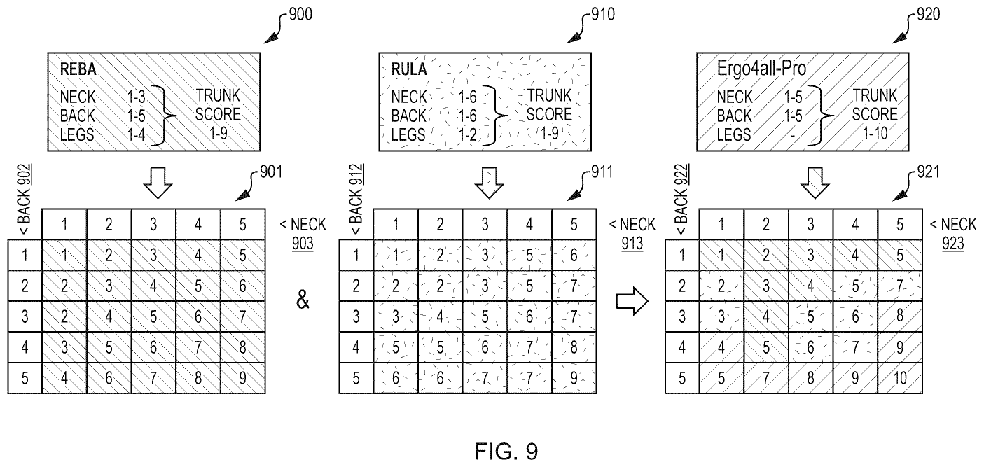

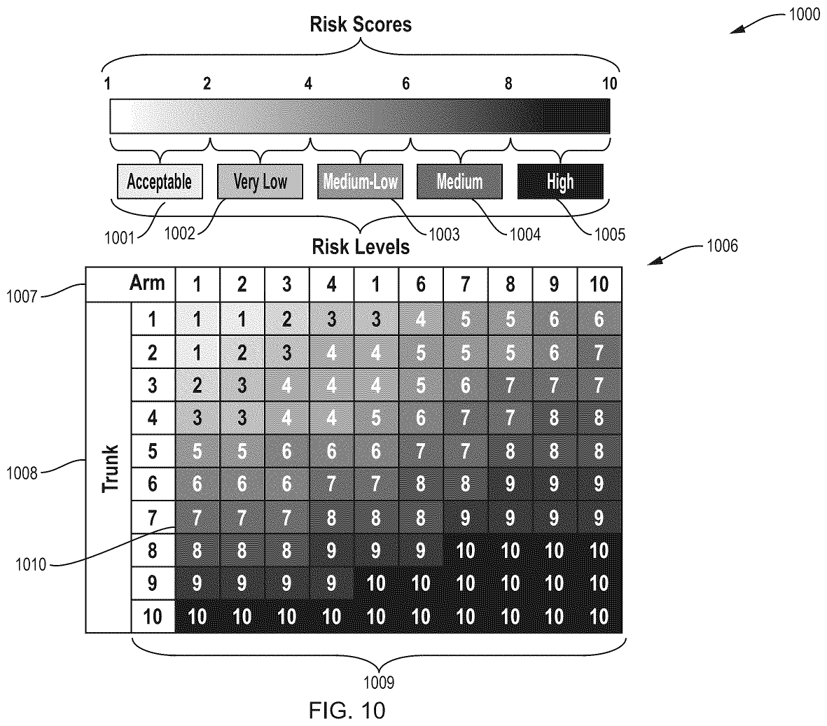

As noted above, after step 313, workflow 300 may alternatively go to step 330 and input a RULA and REBA score at step 331. Following the RULA and REBA input at step 331, the workflow 300 performs fuzzification at step 332 by re-engineering the methods of RULA and REBA. By re-engineering the methods of RULA and REBA, it was determined that risks of the shoulder, elbow, and wrist, should be integrated and presented as risk of an arm, and that the risks of the neck and back should be integrated and presented as risk of a trunk. According to an embodiment, the re-engineering process as applied through the fuzzy rules (i.e., a series of “if” “then” statements) generates the categorization matrix similar to that of RULA and REBA, but is instead customized for DHM systems and elaborated through more sophisticated methods such as OCRA. Thereafter, at step 333, an inference system is used to generates fuzzy rules (optionally using the output from step 325), and defuzzification is used at step 334 to develops a categorization matrix. Thereafter, at step 335 integrated risk for an upper limb is output and the workflow 300 ends at step 336. Steps 332, 333, 334, and 335 of the method 300 are further discussed hereinbelow at least in relation to FIGS. 8A-C, FIG. 9, and FIG. 10.

### Step 310 (FIG. 3): Evaluating Ergonomic Assessment Tools

Ergo4All™ categorizes the risk level of each task in each joint into three levels, similar to a traffic light: for example, green represents low risk, yellow indicates medium risk, and red signifies high risk. Further, it is noted that while color is described, embodiments may instead use shading or any indication of risk. In step 312, OCRA and EAWS, two well-known EATs in assembly line environments, were selected to incorporate insights and enhance the legacy Ergo4All™ method. While OCRA's scoring system is relatively straightforward, EAWS presents a more complex challenge due to the complication of its scoring analysis method. However, prior research by Lavatelli et al. (2012) demonstrates a strong correlation between EAWS4 (a sub-version of EAWS for upper limb evaluation) and OCRA indices, suggesting that understanding OCRA's scoring methodology could provide insights applicable to EAWS4. Therefore, embodiments have identified the logic behind task and operation (a combination of several tasks) evaluation in the OCRA method and generate fuzzy rules accordingly (e.g., at step 323 and/or step 333).

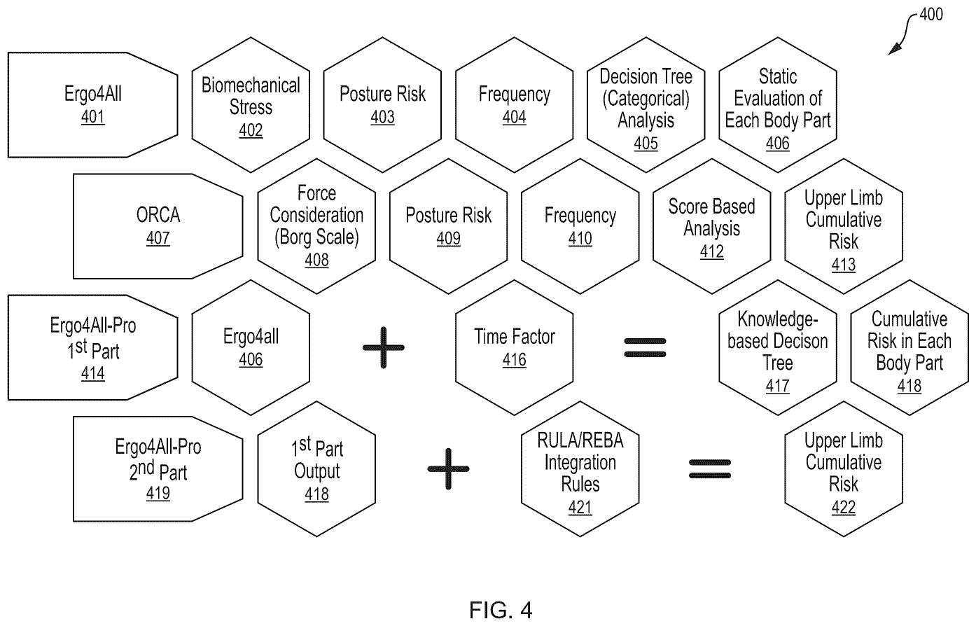

To continue, at step 313, benchmark EAT(s) are selected that typically integrate the risk of individual body parts to generate a unique risk level for the upper limb. Finding this integrated risk enabled embodiments to be validated against existing assessment tools like OCRA or EAWS. FIG. 4, discussed hereinbelow, presents the components of the proposed framework based on initially selected EATs (e.g., OCRA, RULA, REBA).

FIG. 4 is a diagram 400 illustrating various components of legacy EATs, the legacy EAT's respective subcomponents, as well as embodiments disclosed herein, and embodiment's respective subcomponents, that may be used to evaluate cumulative ergonomic risk. Ergo4All™ 401 identifies biomechanical stress 402, posture risk 403, task frequency 404, provides for a categorical analysis 405 and provides for a static evaluation 406 of each body part. OCRA 407 identifies force considerations 408, posture risk 409, task frequency 410, provides for a score based analysis 412, and provides for a cumulative upper limb risk 413. An embodiment, e.g., Ergo4All-Pro™, comprises two parts. The first part 414 takes output from Ergo4All™ (static evaluation of each body party 406), and adds a time factor 416 that each joint spends at each risk level. This provides for a knowledge based decision tree 417 (See FIG. 7), as well as an identification of cumulative risk 418 for each body part. The second part 419 of Ergo4All-Pro™ takes the output of the first part, cumulative risk in each body part 418, and adds RULA/REBA integration rules 421 (e.g., fuzzy rules generated through the re-engineering approach described hereinbelow at least in relation to FIGS. 8A-C and FIGS. 9-11) to determine upper limb cumulative risk 422.

### Step 320 (FIG. 3): Determining Cumulative Risk in Each Body Part

While embodiments integrate time considerations post-assessment of posture and biomechanical risks, OCRA integrates time during the risk evaluation process. Thus, interpreting rules, weights, and processes involving time factor consideration in the OCRA model requires ergonomics professionals' expertise to ensure mathematical validity and acceptability. As presented in FIG. 5, discussed hereinbelow, OCRA integrates time factors into four sections of its scoring system. Embodiments analyze and simulate OCRA's posture section's scoring method and adapt it for the dynamic framework. This approach is applicable due to the shared consideration of specific body parts, such as the shoulder, elbow, and wrist, in both methods, OCRA and embodiments. By understanding the logic behind scoring risk factors for these body parts in OCRA, embodiments generalize it for Ergo4All™ based on time considerations. This multi-step methodology is delineated herein.

FIG. 5 is a flow diagram 500 illustrating the OCRA method and its subcomponents. The OCRA checklist is an ergonomic tool that provides a score based evaluation of ergonomic risk through a checklist that includes several sections. As can be seen in flow diagram 500, the OCRA checklist 511 adds together task frequency 501, required operator force 502 (given by table 515), operator posture 503 (indicated by table 516), and any additional factors 506. Thereafter, the OCRA method 500 multiplies this sum by (i) a recovery multiplier (indicated by table 517) and (ii) a duration multiplier.

### Model Assumptions

As illustrated in FIG. 5, four sections of the OCRA method (force 502, posture 504, recovery multiplier 507, and duration multiplier 509) account for the time factor in determining ergonomic risk scores. While an embodiment utilizes the “posture” 504 analysis of OCRA as the primary portion for development, several assumptions in the sections of frequency 501, additional factors 506, and recovery multiplier 507 should also be considered in defining proper conditions for the reverse engineering process in order to prevent any discrepancies in the generation of fuzzy rules utilized by an embodiment. Below are example assumptions according to an embodiment:

Assumption 1: In the “frequency” 501 section of OCRA, the frequency 501 of technical actions is evaluated based on the number of actions per minute. In an embodiment of the present invention, it is assumed that this parameter is less than 32.4, resulting in a risk score of 0, 0.5 or 1, which can be ignored in developing embodiments via the reverse engineering process of OCRA due to its negligible impact on the final risk score.

Assumption 2: The “additional factors” 506 section of OCRA includes nine conditions for assessing physio-mechanical factors and two conditions for socio-organizational factors. In an embodiment, it is assumed that none of these conditions are applicable.

Assumption 3: The “recovery multiplier” 507 presents a number of hours without adequate recovery time. For an embodiment, it is assumed that there is sufficient recovery time, so no hours are without adequate recovery.

Assumption 4: The “duration multiplier” 509 presents a net duration of repetitive work performed during a shift. It is assumed, in an embodiment, that this duration is equivalent to an 8-hour shift (421-480 minutes). This assumption ensures that the duration factor is not applied twice in embodiments, e.g., Ergo4All-Pro, as an embodiment already considers this factor in joint load assessments.

These assumptions allow the impact of environmental and situational factors to be eliminated during the reverse engineering process of OCRA and focusing on the primary ergonomic factors: “Posture” 504 and “Force” 502 when developing embodiments disclosed herein.

### Force Considerations in OCRA Index

To calculate the force multiplier 502 in the OCRA model, the Borg CR-10 scale (Borg, 1990) is used by interviewing workers and asking them to subjectively describe their perceived effort during repetitive tasks. This subjective tool is not suitable for an embodiment, which is designed for a DHM system. To understand the logic behind OCRA's scoring system, an embodiment relies upon explanations in the OCRA index, as detailed in ISO 11228-3. According to this standard, the force multiplier in OCRA based on the Borg scale is comparable with the force level (FB) in EN 1005-3, which is the basis for joint load consideration in Ergo4All™. Therefore, it can be assumed that both evaluations will result in approximately the same output for force evaluation. FIG. 6 illustrates the correspondence between both models, OCRA and Ergo4All™, based on ISO 11228-3 considerations. Moreover, FIG. 6 illustrates that considerations of load impact on the ergonomic risk evaluation in OCRA are compatible with what has been applied in Ergo4All™. Therefore, embodiments can eliminate the impact of load on the cumulative risk, as the impact of load was previously considered in evaluation of each task through Ergo4All™.

As FIG. 6 shows, while there are some differences between OCRA's force considerations and Ergo4All™ 's joint load evaluations, they are compatible with each other. It is worth noting that force evaluation in OCRA is an input for the final formula to calculate the risk score, but in Ergo4All™, force in each joint is evaluated based on EN 1005-3 and EN 1005-4 to find the final risk level of each task. Therefore, the evaluation in FIG. 6 is a mid-process evaluation in OCRA and whole force consideration in Ergo4All™.

In FIG. 6, the comparison between these two methods is explained in three parts, illustrated in visualization 600 of FIG. 6:

Referring to Force Multiplier in OCRA index 602, in both the OCRA index and Ergo4All™, as can be seen by step 1 603, F_{M} (Force Multiplier) and FB (Maximal Isometric Force), respectively, are multipliers in the denominator of the formula for calculating the risk. In the first step 603, all optimal conditions that prevent increasing the risk level are considered as the optimum value equal to 1. These conditions are FB (maximal isometric force) 611, mv (velocity multiplier) 612, mf (frequency multiplier) 613, and ma (duration multiplier) 614. As mentioned in ISO 11228-3, all the optimal conditions are based on the EN 1005-3 and EN 1005-4 standards (601), making both OCRA index 602 and Ergo4All™ compatible under these conditions.

Still referring to FIG. 6, for Ergo4All™, two standards 601 (EN 1005-3 and 1005-4) are applied to evaluate the load risk in each task. Therefore, these two standards 601 are comparted to the Borg scale applied in OCRA in order to determine their similarities and differences, if any. In Ergo4All™, the load on each joint is evaluated separately from posture risk, with all effective factors such as the duration, frequency, or speed of tasks considered in this part to assess the risk multiplier (mₜ), which ranges from 0 to 1, and the joint load result is reported directly. In OCRA, if one or some of the optimal conditions are not met, the force multiplier (F_{M}) is determined by applying the average level of force as a function of time.

In step 2 604 of FIG. 6, the minimum value of the F_{M} in OCRA is equal to 0.01 when the required force for executing a task exceeds 50% of maximum voluntary contraction (MVC) or is greater than 5 on the Borg scale and is applied for more than 10% of the CT.

In step 3 605 of FIG. 6, referring now to the standards 601, although the OCRA index explains the minimum and maximum values of F_{M} as a percentage of time that force is applied during each CT, other values in this range are not detailed. However, as shown by 615a-b, the duration multiplier in EN 1005-4 specifies three levels for different task durations as a percentage of CT.

Analyzing how OCRA integrates the time duration factor in its evaluation enabled simplification of the OCRA method and elimination of all parts that have already been considered in Ergo4All™, allowing embodiments to concentrate on the most important part(s) of risk evaluation in OCRA. For example, in OCRA, an important part is evaluation of risk as it relates to posture of the operator, particularly when the posture risk evaluation concludes in an awkward posture. In Ergo4All™ tasks involving awkward posture are simulated to be medium and high risk tasks. As illustrated by these three points 603, 604, and 605 in FIG. 6, despite some differences in the process of evaluating the force factor in Ergo4All™ and the OCRA index 602, both yield approximately the same output. The load risk consideration in OCRA and Ergo4All™ are very similar, as such, embodiments consider time as it relates to posture risk.

### Posture Risk in OCRA Checklist

As previously discussed, by applying several assumptions and eliminating similar portions of OCRA and Ergo4All™ from an embodiment, it was found that in order to embody the time factor consideration such an embodiment emphasizes the posture section of OCRA that considers time factor in detail. As such, all considerations in force evaluation in the final OCRA formula have been employed in the joint load evaluation of Ergo4All™. To better apply the reverse engineering approach in analyzing OCRA's risk assessment methodology, the discussion below focuses on posture risk evaluation, aiming to incorporate time factors to develop cumulative risk for each body part.

### Normalization of Scores

Unlike OCRA's scoring approach, Ergo4All™ implements a categorical method, meaning a method that indicates the risk levels in qualitative (e.g., verbal) formats such as “low,” medium,” and “high.”. Therefore, normalization of scores is necessary to establish consistent risk levels across different body parts. TABLE I below facilitates this normalization by scaling OCRA checklist indices between 0 and 1 for uniform interpretation.

TABLE I
Normalized OCRA Score in the Range 0 to 1

| Risk Level | Risk Category | OCRA Score | Normalized Score |
|---|---|---|---|
| Green | Acceptable | <7.5 | 0-0.25 |
| Yellow | Very Low | 7.6-11.0 | 0.26-0.36 |
| Light red | Medium-low | 11.1-14.0 | 0.37-0.46 |
| Dark red | Medium | 14.1-22.5 | 0.47-0.75 |
| Purple | High | ≥22.6 | 0.76-1 |

### Integration of Time-Based Rules in Ergo4All™

Embodiments disclose a method that enhances the static nature of legacy methods, such as Ergo4All™ that considers risk associated with performing one task, by instead evaluating the cumulative risk associated with performance of several tasks. An embodiment achieves this objective by considering time in the evaluation process in order to integrate the durational effect on risk levels.

While OCRA focuses solely on awkward postures in the posture section (504 FIG. 5), Ergo4All™ identifies medium-risk and high-risk tasks by considering additional factors beyond time. Consequently, time-based rules derived from OCRA are selectively applied to medium and high-risk tasks in Ergo4All™ through a reverse engineering approach. According to an embodiment, the output of this reverse engineering approach (See 417 of FIG. 4) are the “fuzzy rules” (See 333 of FIG. 3) represented by the method 700 of FIG. 7 discussed hereinbelow.

In the OCRA checklist for evaluating awkward postures of each body part (516 FIG. 5), including shoulder, elbow, wrist, and hand, the percentage of time in each CT that those postures occurred is calculated, and specific scores are assigned based on the time percentage range. Therefore, for each body part, T(x) (i.e., the percentage of time each worker spends on doing tasks in each risk level) is calculated based on the percentage of cumulative time of awkward posture (medium risk, Tₘ, or high risk, Tₕ), as per Equations (1), (2), and (3) below:

$$T ( x ) = \frac{\sum {t}_{a w k w a r d}}{C T} \qquad ( 1 )$$

$${T}_{m} = \frac{\sum {t}_{medium - risk}}{C T} \qquad ( 2 )$$

$${T}_{h} = \frac{\sum {t}_{high - risk}}{C T} \qquad ( 3 )$$

### Differentiation Between Medium-Risk and High-Risk Tasks

OCRA considers “awkward posture” as one type of so called, risky tasks, in order to illustrate posture related risks. In Ergo4All™, both medium risk and high risk tasks are considered as “awkward postures.” Discussed hereinbelow is an explanation of the differences between medium and high risk tasks as they relate to embodiments disclosed herein.

To align with OCRA's posture evaluation, OCRA's awkward postures were interpreted in a way that includes both medium-risk (Tₘ) and high-risk (Tₕ) tasks in Ergo4All™. Using the shoulder as an example (See, TABLE II, below), medium-risk tasks are evaluated based on normalized score categorizations. Subsequently, risk levels for medium-risk tasks are adjusted to reflect the comprehensive evaluation conducted by Ergo4All™. Differentiation between medium-risk and high-risk tasks is achieved by imposing stricter criteria for the percentage of cumulative time to determine cumulative risk levels. For instance, if the cumulative time of medium-risk level tasks (Tₘ) contains 25% to 50% of the CT, the risk level is “very low”, but if the same amount of time occurs for high-risk tasks (Tₕ), the cumulative risk level is “medium-low”.

TABLE II
Cumulative Risk of the Shoulder Based on Normalized OCRA Scores

| | | | Initial | Adjusted | Concluded |
|---|---|---|---|---|---|
| | OCRA | Norm | Risk | Risk | Risk |
| T(x) | Score | Score | (M_{Ergo4All}) | (M_{Ergo4All}) | (H_{Ergo4All}) |
| T ≤ 10% | 0 | 0 | No Risk | No Risk | Acceptable |
| 10% < T ≤ 25% | 2 | 0.07 | Acceptable | Acceptable | Very Low |
| 25% < T ≤ 50% | 6 | 0.2 | Acceptable | Very Low | Medium-Low |
| 50% < T ≤ 80% | 12 | 0.4 | Medium-Low | Medium-Low | Medium |
| 80% < T | 24 | 0.8 | High | High | High |

This methodology can be applied to the elbow and wrist, as shown in TABLE III, below. However, it appears that the OCRA scoring system for wrist and elbow posture risk evaluation reflects the lower importance of the wrist and elbow compared to the shoulders in the final ergonomics evaluation of OCRA. Since a goal in developing embodiments is to evaluate cumulative risk in each body part, it is important to consider the time factor in greater detail. Therefore, the time consideration in shoulder posture (TABLE II) is applied as a baseline for other body parts, including the neck, elbow, wrist, and back. Additionally, OCRA primarily addresses awkward postures in specific body parts, such as the shoulder, elbow, wrist, and hand. Based on the high correlation between OCRA and EAWS4 (Lavatelli et al., 2012), insights obtained from OCRA's methodology can be generalized to body parts not explicitly considered, such as the back and neck. This extension ensures a holistic approach to ergonomic risk assessment within Ergo4All™. Thus, in the initial step of an embodiment, the time consideration for the shoulder is applied to other body parts, including the neck, elbow, wrist, and back.

TABLE III
Cumulative Risk of Elbow and Wrist
Based on Normalized OCRA scores

| | OCRA | Norm | Risk | Risk |
|---|---|---|---|---|
| T(x) | Score | Score | (M_{Ergo4All}) | (H_{Ergo4All}) |
| T ≤ 25% | 0 | 0 | No Risk | Acceptable |
| 25% < T ≤ 50% | 2 | 0.2 | Acceptable | Medium-Low |
| 50% < T ≤ 80% | 4 | 0.4 | Medium-Low | Medium |
| 80% < T | 8 | 0.8 | High | High |

### Decision Tree for Various Scenarios

While TABLE II and TABLE III present a limited number of scenarios focusing on medium-risk or high-risk tasks, real-world scenarios often entail diverse combinations of task assignments. Hence, TABLE IV, below, can be generated to encompass various combinations of medium and high-risk tasks at a single workstation.

TABLE IV
Different Scenarios of Cumulative Risk for Each Body Part

| | | | Time Zone | | | | |
|---|---|---|---|---|---|---|---|
| | | | 1 | 2 | 3 | 4 | 5 |
| | Time | | H_{Ergo4All} | | | | |
| T (x) | Zone | M_{Ergo4All} | A | VL | ML | M | H |
| T ≤ 10% | 1 | NR | A | VL | ML | M | H |
| 10% < T ≤ 25% | 2 | A | A | VL | ML | M | H |
| 25% < T ≤ 50% | 3 | VL | VL | ML | M | H | H |
| 50% < T ≤ 80% | 4 | ML | ML | M | H | I | I |
| 80% < T | 5 | H | H | H | H | I | I |

In the Fuzzy Inference Systems (FISs) presented in FIG. 3, ergonomic experts' knowledge was utilized to interpret possible cumulative ergonomic risks for each worker and generate fuzzy rules. According to an embodiment, these fuzzy rules are “If . . . , Then . . . ” statements that evaluate specific conditions to derive conclusions using fuzzy logic (Ghorbani et al., 2024b). TABLE V, below, presents the information from TABLE IV in the form of fuzzy rules.

TABLE V
Fuzzy Rules for Interpreting Cumulative Risk in Each Body Part

| | | | | | Cumulative |
|---|---|---|---|---|---|
| | 1^{st} Condition | | 2^{nd} Condition | | Risk |
| If | Tₕ ≤ 10% | & | Tₘ ≤ 25% | Then | Acceptable |
| | | | 25% < Tₘ ≤ 50% | | Very Low |
| | | | 50% < Tₘ ≤ 80% | | Medium Low |
| | | | 80% < Tₘ | | High |
| If | 10% < Tₕ ≤ 25% | & | Tₘ ≤ 25% | Then | Very Low |
| | | | 25% < Tₘ ≤ 50% | | Medium Low |
| | | | 50% < Tₘ ≤ 80% | | Medium |
| | | | 80% < Tₘ | | High |
| If | 25% < Tₕ ≤ 50% | & | Tₘ ≤ 25% | Then | Medium Low |
| | | | 25% < Tₘ ≤ 50% | | Medium |
| | | | 50% < Tₘ | | High |
| If | 50% < Tₕ ≤ 80% | & | Tₘ ≤ 25% | Then | Medium |
| | | | 50% < Tₘ | | High |
| If | 80% < Tₕ | — | — | Then | High |

To visualize the complexity of cumulative risks across different body parts and task combinations, the decision tree depicted in FIG. 7 serves as a practical tool. This decision tree facilitates the assessment and management of ergonomic risks in real-world scenarios, offering clarity amidst complexity. In the embodiments discussed hereinbelow, a cumulative risk score of “Acceptable” equates to a cumulative risk score of “1” for a given body part (elbow, wrist, shoulder, back, or neck), “Very Low” equates to a cumulative risk score of “2,” “Medium Low” equates to a cumulative risk score of “3,” “Medium” equates to a cumulative risk score of “4,” and “High” equates to a cumulative risk score of “5”. An embodiment utilizes the fuzzy rules from TABLE V (and FIG. 7, discussed hereinbelow) to determine the cumulative ergonomic risk of a joint based on the total time duration the joint spends at each risk level. For example, if the time that a joint of the operator is at high risk is <10% of the total time duration of the tasks, and the same joint of the operator is at a medium risk for between 50% and 80% of the total time duration of the tasks, the resulting risk level for that joint across the tasks is “Medium Low,” which equates to a cumulative risk score of “3” for that joint.

FIG. 7 is a decision tree of a method 700 for determining ergonomic risk in a joint across a plurality of tasks, according to an embodiment. according to an embodiment. The decision tree method 700 of FIG. 7 is a visual representation of the interpretation of the fuzzy rules disclosed in TABLE V. The decision tree 700 begins at step 701, and evaluates if the time of the total CT spent in high-risk tasks (Tₕ) is ≤10%. If the total time spent in high-risk tasks is ≤10%, i.e., “Yes” at step 701, the decision tree moves to step 702. At step 702, the method 700 determines if the time spent at medium-risk (Tₘ) is ≤25% of the total CT time. If the time spent at medium-risk Tₘ is ≤25% of the total CT time, i.e., a “Yes” at step 702, the method 700 determines that the risk level is “Acceptable” 703. If however, the time spent at the medium risk task is not ≤25% of the total CT time, i.e., “No” at step 702, the method 700 moves to step 704. At step 704, the method 700 evaluates if the time spent at medium-risk is 25%<Tₘ≤50% of the total CT time. If the time spent at medium-risk is 25%<Tₘ≤50% of the total CT time, i.e., a “Yes” at step 704, the method 700 determines that the risk level is “Very Low” 705. If however, the time spent at the medium risk task is not 25%<Tₘ≤50% of the total CT time, i.e., “No” at step 704, the method 700 moves to step 706. At step 706, the method 700 evaluates if the time spent at medium-risk is 50%<Tₘ≤80% of the total CT time. If the time spent at medium-risk is 50%<Tₘ≤80% of the total CT time, i.e., a “Yes” at step 706, the method 700 determines that the risk level is “Medium-Low” 707. If however, the percentage of total time spent at the medium risk task is not 50%<Tₘ≤80% of the total CT time, i.e., “No” at step 706, the method 700 determines that the risk level is “High” 708.

Continuing the method 700, if the total time of the CT spent in high-risk tasks (Tₕ) is not ≤10%, i.e., “No” at step 701, the method 700 moves to step 710 and evaluates if time of the CT spent in high-risk tasks (Tₕ) is 10%<Tₕ≤25%. If the total time spent in high-risk tasks is 10%<Tₕ≤25%, i.e., “Yes” at step 710, the decision tree moves to step 711. At step 711, the method 700 evaluates if the time spent at medium-risk (Tₘ) is Tₘ≤25% of the total CT time. If the time spent at medium-risk is Tₘ≤25% of the total CT time, i.e., a “Yes” at step 711, the method 700 determines that the risk level is “Very Low” 712. If however, the time spent at the medium risk task is not ≤25% of the total CT time, i.e., “No” at step 711, the method 700 moves to step 713. At step 713, the method 700 determines if the time spent at medium-risk is 25%<Tₘ≤50% of the total CT time. If the time spent at medium-risk is 25%<Tₘ≤50% of the total CT time, i.e., “Yes” at step 713, the method 700 determines that the risk level is “Medium-Low” 714. If however, the time spent at the medium risk task is not 25%<Tₘ≤50% of the total CT time, i.e., “No” at step 713, the method 700 moves to step 715. At step 715, the method 700 determines if the time spent at medium-risk is 50%<Tₘ≤80% of the total CT time. If the time spent at medium-risk is 50%<Tₘ≤80% of the total CT time, i.e., “Yes” at step 715, the method 700 determine the risk level is “Medium” 716. If however, the time spent at the medium risk task is not 50%<Tₘ≤80% of the total CT time, i.e., “No” at step 715, the method 700 determines that the risk level is “High” 717.

Still referring to FIG. 7, if the total time of the CT spent in high-risk tasks (Tₕ) is not 10%<Tₕ≤25%, i.e., “No” at step 710, the method 700 moves to step 720 and determines if time of the total CT spent in high-risk tasks (Tₕ) is 25%<Tₕ≤50%. If the total time spent in high-risk tasks is 25%<Tₕ≤50%, i.e., “Yes” at step 720, the decision tree moves to step 721. At step 721, the method 700 determines if the time spent at medium-risk (Tₘ) is Tₘ≤25% of the total CT time. If the time spent at medium-risk is Tₘ≤25% of the total CT time, i.e., “Yes” at step 721, the method 700 determines the risk level is “Medium-Low” 722. If however, the time spent at the medium risk task is not ≤25% of the total CT time, i.e., “No” at step 721, the method 700 moves to step 723. At step 723, the method 700 evaluates if the time spent at medium-risk is 25%<Tₘ≤50% of the total CT time. If the time spent at medium-risk is 25%<Tₘ≤50% of the total CT time, i.e., “Yes” at step 723, the method 700 determines the risk level is “Medium” 724. If however, the time spent at the medium risk task is not 25%<Tₘ≤50% of the total CT time, i.e., “No” at step 723, the method 700 determines that the risk level is “High” 725.

Continuing with the method 700 of FIG. 7, if the total time of the CT spent in high-risk tasks (Tₕ) is not 25%<Tₕ≤50%, i.e., “No” at step 720, the method 700 moves to step 730 and evaluates if time of the total CT spent in high-risk tasks (Tₕ) is 50%<Tₕ≤80%. If the total time spent in high-risk tasks is 50%<Tₕ≤80%, i.e., “Yes” at step 730, the decision tree moves to step 731. At step 731, the method 700 evaluates if the time spent at medium-risk (Tₘ) is Tₘ≤ 25% of the total CT time. If the time spent at medium-risk is Tₘ≤25% of the total CT time, i.e., “Yes” at step 731, the method 700 determines that the risk level is “Medium” 732. If however, the time spent at the medium risk task is not <25% of the total CT time, i.e., “No” at step 731, the method 700 determines the risk level is “High” 733. If however, the total time of the CT spent in high-risk tasks (Tₕ) is not 50%<Tₕ≤80%, i.e., “No” at step 730, the method 700 determines that the risk level is “High” 740.

### Identifying Upper Limb Ergonomic Risk

Evaluating the cumulative risk in each body part is a novelty of embodiments disclosed herein. However, in addition to evaluating the cumulative risk in each body part individually, it is also helpful to determine cumulative ergonomic risk for a grouping of body parts, e.g., the upper limb. Evaluating ergonomic risk for a grouping of body parts also enables validating embodiments by comparing the output of embodiments with the results of other methods like OCRA, EAWS, etc.

An embodiment determines cumulative ergonomic risk for a grouping of body parts by integrating risk scores of various individual body parts in the upper limb based on RULA and REBA models. To determine rules, according to an embodiment, for these integrations, a reverse engineering approach was conducted, and fuzzy rules were generated based on ergonomic experts' knowledge and presented in matrix form, as shown in FIGS. 8A-8C, 9 and 10, discussed hereinbelow. According to an embodiment, the process of developing an integrated risk score for evaluating the overall risk of the upper limb begins with assigning risk scores from 1 to 5 to each risk level, from “acceptable” to “high,” respectively. Then the following three steps may be conducted:

FIGS. 8A-8C illustrate Step 1 for integrating risk scores according to an embodiment to determine ergonomic risk for an upper limb, composed of an arm and upper trunk. Step 1 includes obtaining a total risk score of an arm. In an embodiment, both arms may be scored, and the highest scoring of the two arms may be used for calculation. The first step involves integrating risk scores for the entire arm, including wrist, elbow, and shoulder. FIGS. 8A-8C illustrate this evaluation in detail, where FIG. 8A illustrates functionality for determining cumulative risk score for the wrist and elbow, according to an embodiment, FIG. 8B illustrates functionality for implementing the cumulative risk score of the elbow and wrist into a matrix for determining cumulative ergonomic risk for an upper limb, according to an embodiment, and FIG. 8C illustrates functionality for the final implemented cumulative risk score of the elbow and wrist into a single matrix, according to an embodiment.

According to RULA and REBA, in arm evaluation, shoulders take precedence over wrist and elbow. Thus, as shown in formula 800 FIG. 8A, the risk scores for the wrist and elbow 801 are combined and converted into cumulative score 807 on a 1 to 5 scale (instead of 1 to 10) to focus on the shoulder in this phase. According to the formula 800: if the combined risk score for the wrist and elbow 801 is 2 or 3 (802) the cumulative risk score 807 is 1; if the combined risk score for the wrist and elbow 801 is 4 or 5 (803) the cumulative risk score 807 is 2; if the combined risk score for the wrist and elbow 801 is 6 or 7 (804) the cumulative risk score 807 is 3; if the combined risk score for the wrist and elbow 801 is 8 or 9 (805) the cumulative risk score 807 is 4; and if the combined risk score for the wrist and elbow 801 is 10 (809) the cumulative risk score 807 is 5.

FIG. 8B is a matrix 810 illustrating the scoring details for determining combined scores 815 of ergonomic risk in the arm using shoulder scores 811 and combined elbow and wrist scores 814 (where the combined elbow and wrist scores 814 are determined using the formula 800 of FIG. 8A and the individual elbow scores 812 and individual wrist scores 813). The matrix 810 serves as a look-up table where the ergonomic risk score for the arm is indicated by the meeting of the shoulder score 811 and combined elbow and wrist score 814. For instance, if the shoulder score is 5 and the combined elbow and wrist score is 3, the ergonomic risk score for the arm is 8, as shown by, e.g., point 816 in matrix 810.

Finally, FIG. 8C presents the final matrix 820 of integrated risk scores for the whole arm, i.e., elbow 821, wrist 822 and shoulder 823. In FIG. 8C, the details about integrating risk scores of the elbow and wrist, as illustrated in 814 of FIG. 8B, are omitted, thereby showing only the final scores for elbow 821 and wrist 822.

FIG. 9 illustrates Step 2 for determining cumulative ergonomic risk for a grouping of body parts, e.g., upper limb, as part of the re-engineering process of RULA and REBA. Step 2 determines a total risk score of trunk. This step integrates risk scores of the neck and back from existing methods, e.g., REBA 900 and RULA 910 to produce a 10-point score for the trunk 920 as indicated by the matrix 921. The REBA method 900 determines trunk scores from 1-9 for the back 902 and neck 903, as illustrated by matrix 901. The RULA method 910 determines trunk scores from 1-9 for the back 912 and the neck 913, as illustrated by matrix 911. An embodiment, e.g., Ergo4All-Pro™ 920 determines trunk scores from 1-10 for the neck 923 and back 922, as illustrated by the matrix 921, based on a combination of the matrix 901 from REBA and the matrix 911 from RULA. Since REBA 900 and RULA 910 also consider the legs in the posture risk evaluation for the trunk, and use different score ranges for the back and neck, each method is evaluated separately and covered in FIG. 9. Therefore, the final matrix of integrated risk scores 921 for the trunk is evaluated based on ergonomists' knowledge to address the differences between the final proposed model 920 and RULA 910 or REBA 900, which evaluate posture risk in an integrated manner.

FIG. 10 illustrates Step 3 for determining cumulative ergonomic risk for a grouping of body parts, e.g., upper limb, according to an embodiment. Step 3 determines a final risk score of upper limb based on the determined ergonomic risk of the arm (as shown and explained herein in relation to FIGS. 8A-C) and the determined ergonomic risk of the trunk (as shown and explained herein in relation to FIG. 9). According to an embodiment, the ergonomic risk scores 1009 for the upper limb are shown by the matrix 1006 and are based on both the arm scores 1007 and trunk scores 1008. The matrix 1006 serves as a look-up table that indicates upper limb scores 1009 as a function of arm scores 1007 and trunk scores 1008. For instance, if the arm risk 1007 is 1 and the trunk risk 1008 is 7, the upper limb score 1009 is 57, given by entry 1010 in matrix 1006. As shown in FIG. 10, the upper limb scores 1009, according to such an embodiment, can be shown by scale 1000 and can be categorized into five risk levels. These five risk levels may be a score between 1 and 2 being “Acceptable” 1001, a score between 2 and 4 being “Very Low” 1002, a score between 4 and 6 being “Medium-Low” 1003, a score between 6 and 8 being “Medium” 1004, and a score between 8 and 10 being “High” 1005.

Although embodiments have several considerable differences compared to conventional methods, the results from embodiments are comparable to several methods that provide an integrated risk level for the upper limb or the whole body, such as EAWS, OCRA, etc.

### Example Model Implementation

Herein, to validate embodiments, a real sample workstation is evaluated, and the results are compared with benchmark EATs. Additionally, embodiments are applied to several synthesized scenarios, and the results are discussed to validate the embodiments.

Implementing an embodiment on a real case study and several synthesized scenarios demonstrates ability of embodiments to investigate potential risks in various upper limb body parts, both individually and in an integrated manner. Although this new feature is crucial for enhancing DHM systems and the ergonomic design of workplaces in virtual environments, the validation process requires certain considerations. As explained in previous sections, to compare the results of embodiments with other benchmark EATs, a scoring system was implemented to incorporate RULA and REBA methodologies in assessing the integrated risk of the upper limb. While the base of an example embodiment, Ergo4All™, is a categorical tool, and the evaluation of cumulative risk in each body part is also categorical, the final output of an example embodiment for upper limb risk includes both categorical and score-based assessments. However, in such an embodiment each category in the upper limb results contains two scores, simplifying the evaluation of the embodiment's convergence with benchmarks. These two scores are sufficient to show the approximate alignment of the integrated upper limb risk with traditional EATs, given the various imprecisions inherent in the virtual design phase compared to real workplaces.

### A Real Case Study

In this section a real assembly workstation from a car manufacturer is evaluated using OCRA, EAWS, EAWS4, and an embodiment. The results are then compared to determine if the embodiment of the present invention is validated. TABLE VI, below, presents the operations, tasks, and execution time of each task in the workstation being evaluated. In addition, based on a video of this example workstation, the risk level in various body parts is evaluated using Ergo4All™, as shown in TABLE VI. In this case study, the CT is equal to 60 seconds. Therefore, the cumulative time of medium and high-risk tasks in each CT, Tₘ and Tₕ, can be calculated using Equations (2) and (3), respectively. Then, according to the decision tree in FIG. 7, the cumulative risk in each joint is assessed using an embodiment and the results are presented in TABLE VII, below.

TABLE VI
Detailed Information of the Sample Workstation with Ergonomic
Assessment of Each Task Through Ergo4All ™

| | | | | Shoulder | | Elbow | | Wrist | |
|---|---|---|---|---|---|---|---|---|---|
| Operation Tasks | Time | Back | Neck | Right | Left | Right | Left | Right | Left |

1. Prepare component A

| | | | | | | | | | |
|---|---|---|---|---|---|---|---|---|---|
| 1.1 Retrieve Component A | 2 | L | L | L | H | L | L | L | L |
| 1.2 Align Component A on Surface | 2 | L | H | L | L | L | L | L | L |
| 1.3 Position Face Up on Fixture | 1 | L | H | L | L | L | L | L | L |

2. Install Component B to A

| | | | | | | | | | |
|---|---|---|---|---|---|---|---|---|---|
| 2.1 Retrieve Component B | 1 | L | L | L | L | L | L | L | L |
| 2.2 Align Component B to A | 3 | L | H | L | L | L | L | L | L |
| 2.3 Attach Component B to A | 2 | L | H | L | L | L | L | L | L |

3. Secure Component B to A with screws

| | | | | | | | | | |
|---|---|---|---|---|---|---|---|---|---|
| 3.1 Collect Screws and Load Tool | 2 | L | H | L | L | L | L | L | L |
| 3.2 Fasten Top Left Screw | 3 | L | H | H | L | M | L | L | L |
| 3.3 Fasten Top Right Screw | 3 | L | H | L | L | L | L | L | L |
| 3.4 Fasten Bottom Left Screw | 2 | L | H | L | L | L | L | L | L |
| 3.5 Fasten Bottom Right Screw | 2 | L | H | L | L | L | L | L | L |
| 3.6 Fasten Top Middle Screw | 3 | L | L | L | L | L | M | L | L |
| 3.7 Fasten Bottom Middle Screw | 2 | L | H | L | L | L | L | L | L |

4. Verify Documentation

| | | | | | | | | | |
|---|---|---|---|---|---|---|---|---|---|
| 4.1 Verify Assembly Document | 1 | L | H | L | L | M | L | L | L |

5. Verify Documentation

| | | | | | | | | | |
|---|---|---|---|---|---|---|---|---|---|
| 5.1 Scan Component B Barcode & | 2 | L | L | L | L | L | L | L | L |
| Confirm | | | | | | | | | |

6. Connect Coupler to Component B

| | | | | | | | | | |
|---|---|---|---|---|---|---|---|---|---|
| 6.1 Position Coupler Near Opening | 3 | L | H | L | L | L | L | L | M |
| 6.2 Align Coupler with B | 1 | L | H | L | L | L | L | L | M |
| 6.3 Connect Coupler Securely | 1 | L | H | H | L | L | L | L | L |

7. Route Harness Through Component A

| | | | | | | | | | |
|---|---|---|---|---|---|---|---|---|---|
| 7.1 Route Harness Through Opening | 1 | L | H | L | L | L | L | L | L |

8. Route Branch Through Frame & Component A

| | | | | | | | | | |
|---|---|---|---|---|---|---|---|---|---|
| 8.1 Route Branch Through Frame to | 2 | L | H | L | L | L | L | L | L |
| Front | | | | | | | | | |

9. Install Component A to Frame

| | | | | | | | | | |
|---|---|---|---|---|---|---|---|---|---|
| 9.1 Align Component A to frame | 2 | L | L | L | L | L | L | L | L |
| 9.2 Secure Right Side | 1 | L | H | L | L | L | L | L | M |
| 9.3 Secure Left Side | 4 | L | L | L | L | M | L | L | L |
| 9.4 Secure Middle Area | 4 | L | L | L | L | L | L | L | M |
| 9.5 Secure Above Component | 3 | L | L | L | H | L | L | L | L |
| Downward | | | | | | | | | |
| 9.6 Secure Above Component | 1 | L | L | L | L | L | L | L | L |
| Upward | | | | | | | | | |

TABLE VII
Results of Cumulative Risk in Each Body Part Based
on Embodiment, e.g., Ergo4All-Pro ™

| Cumulative Time of | | | Shoulder | | Elbow | | Wrist | |
|---|---|---|---|---|---|---|---|---|
| Risky Tasks (% of CT) | Back | Neck | Right | Left | Right | Left | Right | Left |
| Tₘ | 0 | 0 | 0 | 0 | 14 | 7 | 0 | 14 |
| Tₕ | 0 | 53 | 7 | 8 | 0 | 0 | 0 | 0 |
| Cumulative Risk | A | M | A | A | A | A | A | A |

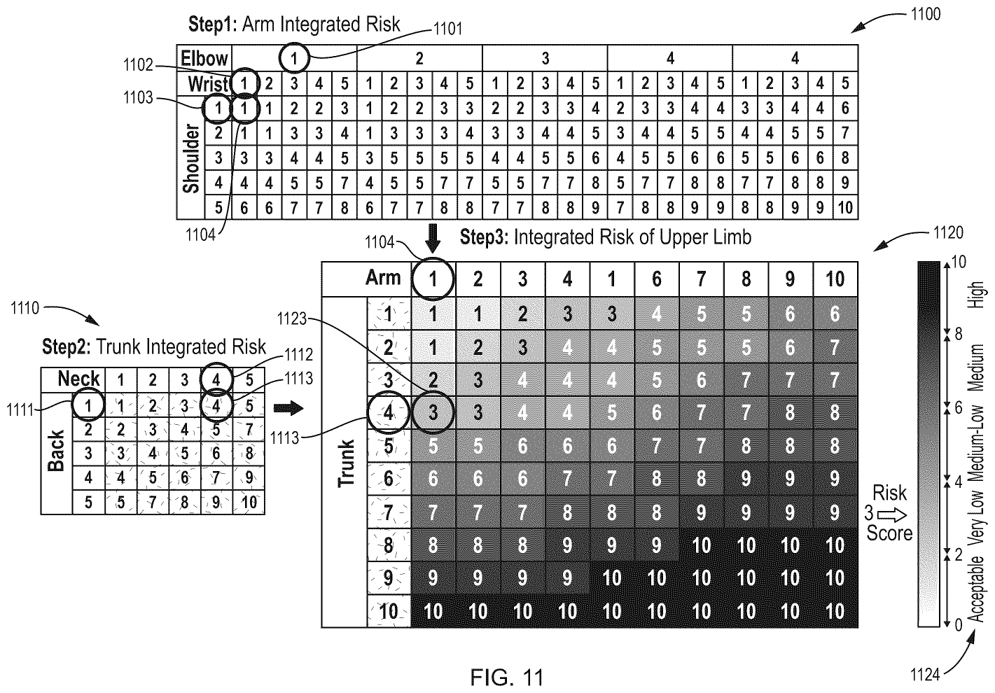

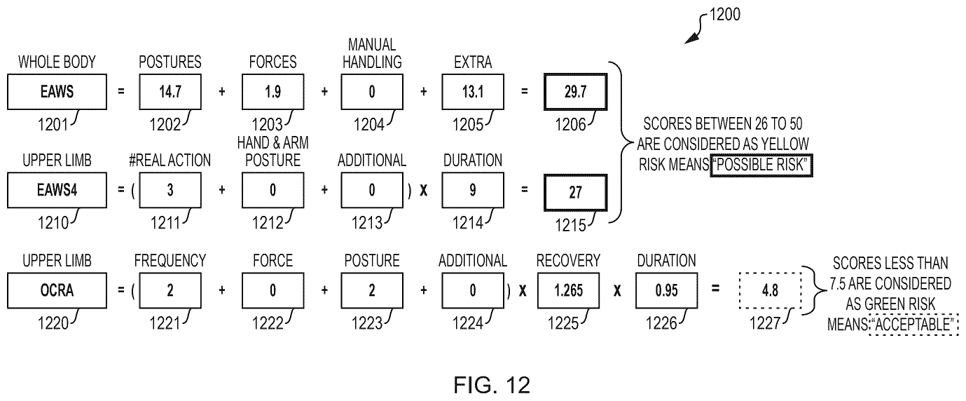

The cumulative risk for the upper limb of the worker in this workstation, based on an embodiment is very low, with a risk score of 3 (See 1123 of FIG. 11). FIG. 11, discussed hereinbelow, provides more details. Furthermore, the ergonomic risk of this sample workstation was evaluated using three different EATs: EAWS, EAWS4, and OCRA, and their procedures are presented in FIG. 12, discussed hereinbelow.

FIG. 11 shows three matrices, the arm integrated risk matrix 1100, the trunk integrated risk matrix 1110, and the integrated risk of upper limb matrix 1120, according to an embodiment. The first step of obtaining the integrated risk of the upper limb, as previously discussed in relation to FIGS. 8A-8C above, is to identify the arm integrated risk 1100. In this example, the operator's elbow joint has a risk score of “1” 1101, the operator's wrist joint has a risk score of “1” 1102, and the operator's shoulder has a risk score of “1” 1103. This results in an integrated arm risk score of “1” 1104. The second step, as previously discussed in relation to FIG. 9 above, is to identify the integrated trunk risk 1110. In this example, the operator's neck joint has a risk score of “4” 1112 and the operator's back has a risk score of “1” 1111. This results in an integrated trunk risk score of “4” 1113. The integrated arm risk score of “1” 1104 and the integrated trunk risk score of “4” 1113, are then combined for the integrated risk of upper limb at step 3, as previously discussed in relation to FIG. 10 above. As can be seen in matrix 1120, the arm risk score of “1” 1104 and the trunk risk score of “4” 1113 results in an integrated risk of upper limb score of “3” 1123. As can be seen by the risk score scale 1124, an integrated risk of upper limb being “3” 1123 may be considered a “Very Low” integrated risk score of the upper limb.

FIG. 12 illustrates the risk evaluation of the example workstation evaluated using an embodiment as described in relation to FIG. 11, but with different benchmark tools, namely EAWS 1201, EAWS4 1210, and OCRA 1220. EAWS 1201 scoring considers the whole body as a sum of: postures 1202 (14.7), forces 1203 (1.9), manual handling 1204 (0), and extra 1205 (13.1), yielding an EAWS score 1206 of 29.7. EAWS4 1210 considers the upper limb as a sum of: real action 1211 (3), hand and arm posture 1212 (0) and additional 1213 (0). This sum is then multiplied by task duration 1214 (9), yielding a EAWS4 score 1215 of 27. OCRA 1220 considers the upper limb as a sum of: frequency 1221 (2), force 1222 (0), posture 1223 (2), and additional 1224 (0). This sum is then multiplied by recovery time 1225 (1.265), and multiplied once more by the task duration 1226 (0.95), yielding an OCRA score 1227 of 4.8. In both EAWS 1201 and EAWS4 1210, a score between 26 and 50 is considered a “yellow” risk or “possible risk” score. In OCRA 1220, a score of less than 7.5 is considered a “green” risk or “Acceptable” score.

By comparing the results of various EATs (as shown in FIG. 12) with the evaluation results from an embodiment (as shown in FIG. 11), it can be concluded that the embodiments aligns more closely with EAWS4, as both the results of the embodiment (1123) in FIG. 11 and EAWS4 (1215) in FIG. 12 returned a “yellow” or “possible risk”/“Very Low” risk score. The results from EAWS (1206) differ slightly because EAWS considers the entire body, whereas embodiments focus only on the upper limb. The differences between OCRA and the embodiment in this specific example are due to the neck risk, which is not considered in the OCRA checklist.

### Scenario-Based Analysis

It is demonstrated hereinabove that the results of an example embodiment are consistent with EAWS4 and more precise than OCRA, as the example embodiment accounts for neck risk in addition to other body parts. To further compare the results of existing EATs with embodiments and to better analyze their differences, weaknesses, and strengths, several scenarios are discussed herein. Furthermore, the results of implementing an embodiment are compared with EAWS4 and OCRA as upper limb EATs.

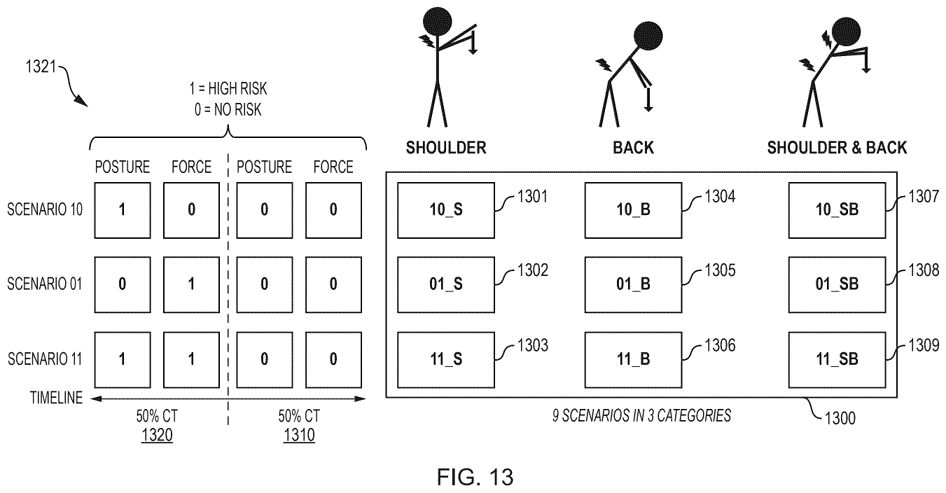

FIG. 13 presents nine scenarios 1300 that focus on the shoulder (1301-1303), back (1304-1306), and back and shoulder (1307-1309). In these scenarios 1301-1309, the same number of tasks and task times as in the example workstation from FIG. 11 are considered. However, it is assumed that the worker in this workstation spends 50% of the CT on tasks with no risk (1310) and the other 50% exposed to high risks (1320) in the shoulders, back, or both. As the proposed methodology, based on Ergo4All™, focuses on posture and force, the scenarios generated consider the risk to shoulders and back in these two forms. The coding system 1321 in FIG. 13 indicates the type of risk in each body part. For instance, scenario 01-B 1305 is a sample where the worker is exposed to a high-risk level of force in their back for one-half of the total CT.

To continue, the scenarios 1300 are evaluated using the method 700. It is noted that according to the method 700 of FIG. 7, for the scenarios 1300, the cumulative risk for the joint(s) under the risk of posture and/or force will be medium-low (Tₕ=50%) (See 722 of FIG. 7). Therefore, in each group of scenarios related to the shoulders, back, or both, there will be no difference between the cumulative risk of the joint under the risk of posture, force, or both. This is because in the method 700 the results are based on the worst-case. Thus, whether the risk is caused by poor posture, high load, or both, the result for the task will be “High”. As a result, it is only necessary to evaluate the integrated risk of the upper limb for each of the three groups of scenarios based on the procedure explained above in relation to FIGS. 8A-8C, FIG. 9, FIG. 10, and FIG. 11.

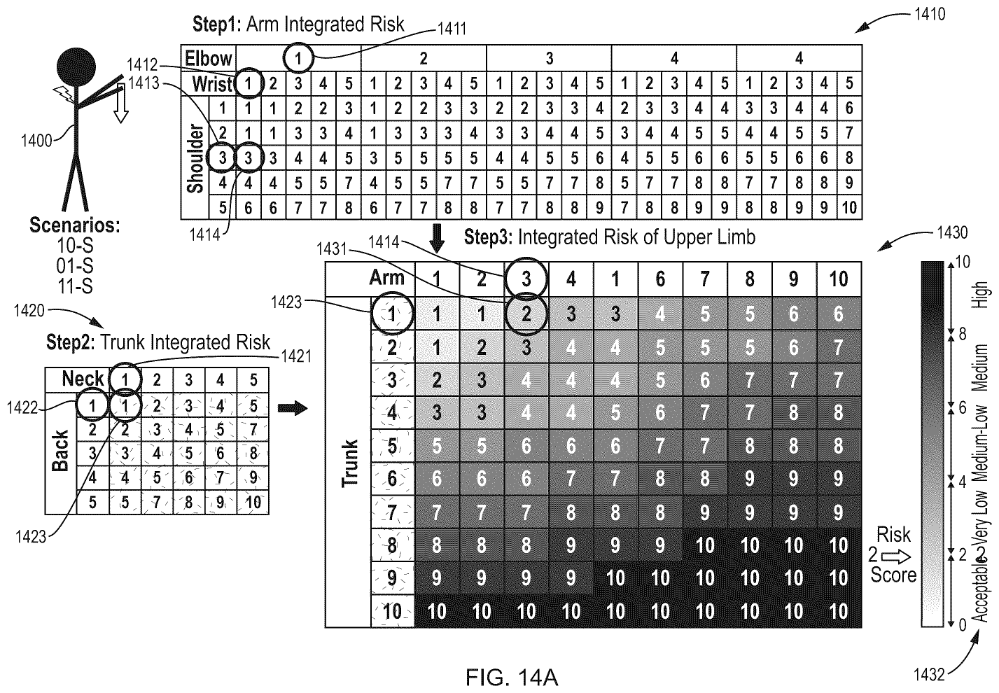

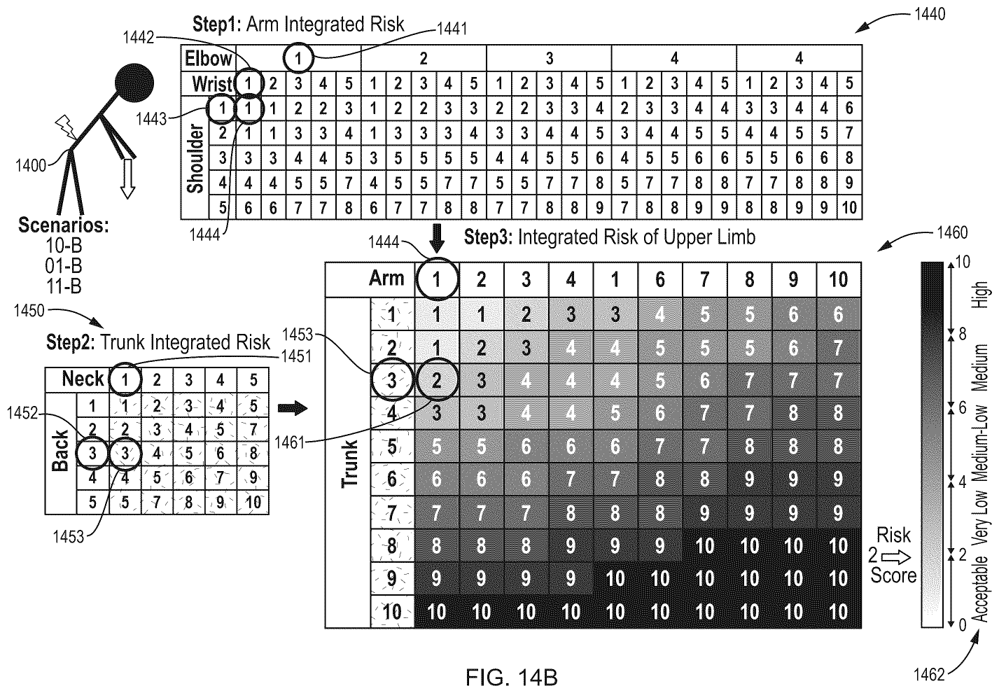

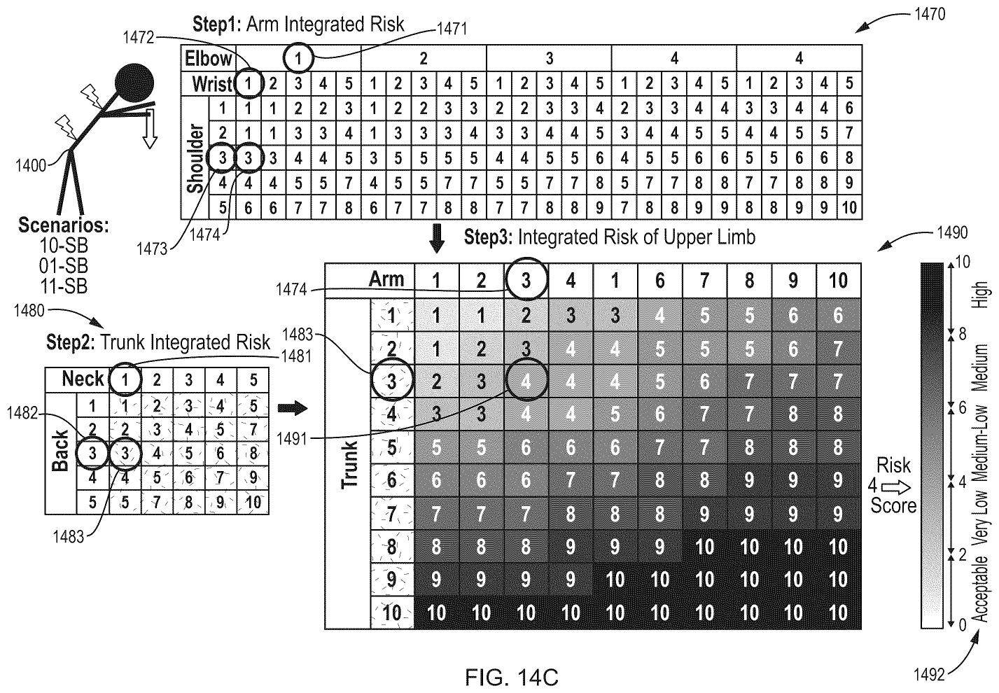

FIGS. 14A-14C illustrate the application of embodiments to evaluate integrated ergonomic risk in the upper limb for each group of scenarios 1300. FIG. 14A illustrates an upper limb risk for the first group of scenarios of FIG. 13, related to risk on the shoulder (1301-1303), according to an embodiment. Following the steps outlined herein above in relation to FIGS. 8A-C, 9, and 10 for this example in FIG. 14A, the first step is to determine an arm integrated risk, shown by matrix 1410. In matrix 1410, the elbow has a risk score of “1” 1411, the wrist has a risk score of “1” 1412, and the shoulder has a risk score of “3” 1413. This yields an arm integrated risk score for the operator 1400 of “3” 1414. Thereafter, the second step is to determine a trunk integrated risk, shown by matrix 1420. In matrix 1420, the neck has a risk score of “1” 1421 and the back has a risk score of “1” 1422. This produces a trunk integrated risk score for the operator 1400 of “1” 1423. To continue, the third step is to determine the integrated risk for the upper limb, shown by matrix 1430. In matrix 1430, the arm integrated risk score is “3” 1414, and the trunk integrated risk score is “1” 1423, thereby yielding an integrated risk score for the upper limb of “2” 1431, which, according to scale 1432 may be considered an “Acceptable” risk score.

FIG. 14B illustrates application of an embodiment to determine integrated ergonomic risk for an upper limb risk for the second group of scenarios of FIG. 13, related to risk on back (1304-1306). Following the steps outlined herein above in relation to FIGS. 8A-C, 9, and 10 for this example in FIG. 14B, the first step is to obtain an arm integrated risk, shown by matrix 1440. In matrix 1440, the elbow has a risk score of “1” 1441, the wrist has a risk score of “1” 1442, and the shoulder has a risk score of “1” 1443, which yields an arm integrated risk score for the operator 1400 of “1” 1444. Thereafter, the second step is to determine a trunk integrated risk, shown by matrix 1450. In matrix 1450, the neck has a risk score of “1” 1451 and the back has a risk score of “3” 1452 to produce a trunk integrated risk score for the operator 1400 of “3” 1453. To continue, the third step is to determine the integrated risk score for the upper limb, shown by matrix 1460. In matrix 1460, the arm integrated risk score is “1” 1444 and the trunk integrated risk score is “3” 1453, thereby producing an integrated risk score for the upper limb score of “2” 1461, which may be considered an “Acceptable” risk score according to the scale 1462.

FIG. 14C illustrates application of an embodiment to determine integrated ergonomic risk for an upper limb for the third group of scenarios of FIG. 13, related to risk on both shoulder and back (1307-1309), according to an embodiment. Following the steps outlined herein above in relation to FIGS. 8A-C, 9, and 10 for this example in FIG. 14C, the first step is to determine an arm integrated risk, shown by matrix 1470. In matrix 1470, the elbow has a risk score of “1” 1471, the wrist has a risk score of “1” 1472, and the shoulder has a risk score of “3” 1473. This produces an arm integrated risk score for the operator 1400 of “3” 1474. Thereafter, the second step is to determine a trunk integrated risk, shown by matrix 1480. In matrix 1480, the neck has a risk score of “1” 1481 and the back has a risk score of “3” 1482. This produces a trunk integrated risk score for the operator 1400 of “3” 1483. To continue, the third step is to determine the integrated risk score for the upper limb, shown by matrix 1490. In matrix 1490, the arm integrated risk score is “3” 1474 and the trunk integrated risk score is “3” 1483, thereby yielding an integrated risk score for the upper limb of “4” 1491, which may be considered an “Low” risk score according to scale 1492.

FIGS. 14A-14C illustrate that in the first two groups of scenarios (FIG. 14A and FIG. 14B), where there is a medium-low level of risk to the shoulder or back, the integrated risk to the upper limb is at an acceptable level with a risk score of “2” 1431 and 1461. Furthermore, when there are medium-low risks to both the shoulder and back, the integrated risk to the upper limb is very low with a risk score of “4” 1491. It is noted that an embodiment may considered primarily a categorical assessment tool in the first step for cumulative risk in each body part, and in the second step, such an embodiment provides a risk score to evaluate the integrated risk of the upper limb. Therefore, in the integrated risk of the upper limb, embodiments can provide scores that are a kind of categorized risk with two scores in each category. For example, when the risk score in the third group of scenarios (FIG. 14C) (risk on both shoulder and back) is “4” 1491, it means that the risk level is “very-low” but closer to “medium-low” than “acceptable”. With this in mind, it is easier to understand how the proposed model can be compared to other score-based methods like EAWS4 and OCRA. TABLE VIII, below, presents the results of the evaluation of all nine scenarios (1301-1309) using an example embodiment and two benchmark methods, OCRA and EAWS4.

TABLE VIII
Results of Integrated Risk of Upper Limb in Benchmark EATs and Ergo4All-Pro ™

| | | Shoulder (S) | | | Back (B) | | | Shoulder & Back (SB) | | |
|---|---|---|---|---|---|---|---|---|---|---|
| Scenario | | | Ergo4All- | | | Ergo4All- | | | Ergo4All- | |
| Posture | Force | OCRA | Pro ™ | EAWS4 | OCRA | Pro | EAWS4 | OCRA | Pro ™ | EAWS4 |
| 1 | 0 | 7.6 | 2 | 25 | 0 | 2 | 5 | 7.6 | 4 | 25 |
| 0 | 1 | 5.1 | 2 | 10 | 5.1 | 2 | 10 | 5.1 | 4 | 10 |
| 1 | 1 | 12.7 | 2 | 30 | 5.1 | 2 | 10 | 12.7 | 4 | 30 |

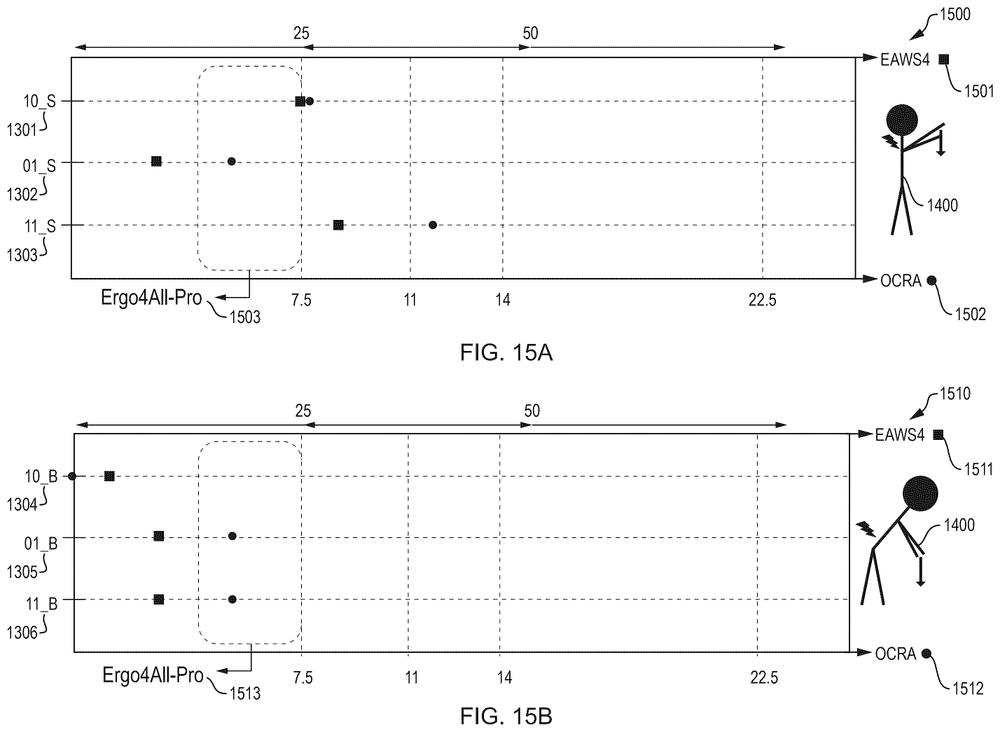

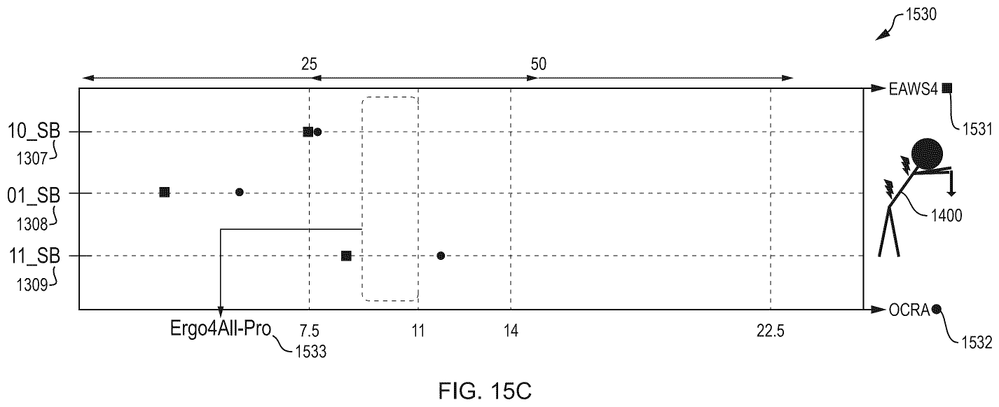

FIGS. 15A-C are charts illustrating a comparison of results of ergonomic evaluations performed using OCRA, EAWS4, and embodiments. FIG. 15A illustrates said comparison for the scenarios related to the risk on the shoulder as illustrated in FIG. 14A, FIG. 15B illustrates said comparison for the scenarios related to the risk on the back as illustrated in FIG. 14B, and FIG. 15C illustrates said comparison for the scenarios related to risk on both the shoulder and the back combined as illustrated in FIG. 14C.

By visualizing the results from EAWS4, OCRA and embodiments in FIGS. 15A-15C, discussed below, it becomes easier to explain and analyze the differences. Comparing the outcomes of these three assessment tools yields the following findings:

As shown in FIGS. 15A and 15C, related to shoulder and combined shoulder and back scenarios, the same results from EAWS4 and OCRA are observed. This indicates that EAWS4 and OCRA may overlook the risk of the back while overestimating the risk to the shoulder. This result is expected from OCRA as it does not consider back risk, focusing instead on the shoulder, elbow, wrist and hand. Therefore, if there is any risk in one of those parts, the integrated risk to the upper limb may be slightly exaggerated. Although OCRA is sensitive to these body parts, in an embodiment of the present invention, these parts are included in the integrated risk of the arm and then combined with the trunk risk, which includes the back and neck. As a result, the final upper limb risk evaluated by such an embodiment is moderated when the risk is present in either the shoulder or back, compared to OCRA and EAWS4. However, in the third group of scenarios where both shoulder and back are at risk, an embodiment provides a different evaluation compared to the first two groups, which seems more logical given the higher risk when both parts are involved.

FIG. 15A is a chart 1500 illustrating the results of evaluating the first group of scenarios related to risk on shoulder (scenarios 1301-1303 FIG. 13), according to an embodiment, EAWS4, and OCRA. The scale for EAWS4 1501 scores can be seen on the top portion of chart 1500, while the scale for OCRA 1502 scores can be seen on the bottom portion of chart 1500. It can be observed that for the 10_S scenario (1301), where the ergonomic risk type for the shoulder is posture, EAWS4 1501, OCRA 1502, and an embodiments determine the risk level to be roughly the same. It can also be observed that for the 01_S scenario (1302), where the ergonomic risk type for the shoulder is force, OCRA 1502 determines the force risk to be slightly higher than the risk determined using EAWS4 1501. For the 11_S scenario (1303), where the ergonomic risk type for the shoulder is both posture and force OCRA 1502 results in an ergonomic risk score that is much higher than the risk score determined using EAWS4 1501. An embodiment, e.g., Ergo4All-Pro™, 1503 provided for reasonable results across all three scenarios.

FIG. 15B is a chart 1510 illustrating the results of evaluating the group of scenarios related to risk on back (1304-1306 FIG. 13), according to an embodiment, EAWS4 and OCRA. The scale for EAWS4 1511 scores can be seen on the top portion of chart 1510, while the scale for OCRA 1512 scores can be seen on the bottom portion of chart 1510. It can be observed that for the 10_B scenario (1304), where the ergonomic risk type for the back is posture, EAWS4 1510, OCRA 1512 determine the risk level to be roughly the same. It can also be observed that for the 01_B scenario (1305), where the ergonomic risk type for the back is force, OCRA 1512 determines the force risk to be slightly higher than does EAWS4 1511. For the 11_B scenario (1306), where the ergonomic risk type for the back is both posture and force OCRA 1512 results in an ergonomic risk score that is higher than the risk score determined using EAWS4 1501. An embodiment, e.g., Ergo4All-Pro™, 1513 provided for reasonable results across all three scenarios.

FIG. 15C is a chart 1520 illustrating the results of evaluating the group of scenarios related to risk on both shoulder and back (1307-1309 FIG. 13), according to an embodiment, EAWS4 and OCRA. The scale for EAWS4 1531 scores can be seen on the top portion of chart 1530, while the scale for OCRA 1532 scores can be seen on the bottom portion of chart 1530. It can be observed that for the 10_SB scenario (1307), where the ergonomic risk type for the shoulder and back is posture, EAWS4 1531, OCRA 1532 determine the risk level to be roughly the same. It can also be observed that for the 01_SB scenario (1308), where the ergonomic risk type for the shoulder and back is force, OCRA 1532 determines the force risk to be slightly higher than does EAWS4 1531. For the 11_SB scenario (1309), where the ergonomic risk type for the shoulder and back is both posture and force OCRA 1532 results in an ergonomic risk score that is higher than the risk score determined using EAWS4 1531. An embodiment, e.g., Ergo4All-Pro™, 1533 provided for reasonable results across all three scenarios.

Although an embodiment is not sensitive to the type of risk, e.g., whether the risk is because of posture or force, such an embodiment does account for the force risk in all scenarios. However, in “01” scenarios where the risk source is force, EAWS4 and OCRA identify the same risk level across all groups, indicating EAWS4 and OCRA may not properly assess force risk.

It is noted that the embodiment evaluated in FIGS. 15A-15C is categorical method and FIGS. 15A-C illustrate that the results of such an embodiment can be evaluated to determine if they are approximately aligned with the other two benchmark tools, EAWS4 and OCRA. Based on the explanations, in all three groups of scenarios, 1301-1303 (shoulder), 1304-1306 (back), and 1307-1309 (shoulder and back), embodiments yield reasonable results.

In the first group of scenarios (FIG. 15A), the example embodiment 1503 provides an appropriate evaluation regardless of whether the risk source is posture or force, and when both posture and force exist the evaluated risk is not exaggerated.

In the second group of scenarios (FIG. 15B), the example embodiment 1513 can approximately identify the risk to the back, while the EAWS4 and OCRA methods, compared to the shoulder (FIG. 15A), tend to ignore the risk to the back, especially when the risk source is posture or both posture and force.

A strength of the example embodiment is evident in the third group of scenarios (FIG. 15C), where there is a risk to both shoulder and back. In these cases, the example embodiment 1533 not only properly considers the risk to the back, but also accounts for the risk resulting from force.

Advantageously, embodiments provide various potential implementations and contributions in academic and industrial environments. Discussed hereinbelow is detailed analysis and discussion of the results obtained from implementing embodiments on a example workstation and scenarios.

### Validation & Verification

The validation and verification of embodiments is important to ensure the reliability and accuracy of embodiments to assess ergonomic risks. Hereinbelow is a discussion on the methods used to validate and verify embodiments, emphasizing alignment between the example embodiments and established EATs and the performance of embodiments across different scenarios.

To validate the accuracy of an embodiment, a real assembly workstation from a car manufacturer was evaluated. The embodiment's outputs were compared with those obtained from three benchmark ergonomic assessment tools: EAWS, EAWS4, and OCRA. As detailed in TABLES VI, VI, and VII above, as well as FIGS. 10 and 11, embodiments provide a more refined evaluation of ergonomic risks, particularly in considering risk factors for the neck and upper limbs. This validation confirms that embodiments align well with EAWS4 and offer a comparable or even more precise risk assessment, especially in areas that other tools may overlook.

Further validation was performed using several synthesized scenarios designed to test embodiments under different ergonomic conditions. These scenarios focused on varying levels of risk exposure to the shoulder and back, as illustrated in FIG. 13. The performance of embodiments was compared with EAWS4 and OCRA, and the results are summarized in FIGS. 15A-C. The scenario-based analysis demonstrates that embodiments consistently provide reasonable and accurate risk assessments, particularly when evaluating complex scenarios involving multiple body parts. The ability of embodiments to assess both posture and force risks across different scenarios underscores its robustness and applicability in diverse ergonomic contexts.

### Example Implementations

Embodiments provide a comprehensive approach to ergonomic risk evaluation in virtual environments, with the capability to assess cumulative risk in each body part, offering significant potential. First, embodiments disclose a novel expert system infrastructure that utilizes reverse engineering to develop fuzzy rules based on ergonomists' knowledge and expertise. This approach is a substantial contribution, enabling sensitivity analyses to identify the most appropriate rules for accurately predicting future ergonomic risks. Embodiments also establish thresholds, e.g., based on expert knowledge, providing a foundation for further exploration. These thresholds can be analyzed to refine fuzzy rules, and alternative expert systems can be employed to adjust these rules using different methodologies, leading to more precise risk evaluations.

Second, embodiments can be used to address various optimization problems, such as assembly line balancing problems (ALBPs) (Ghorbani et al., 2024b), assembly line worker assignment and balancing problems (ALWABPs) (Ghorbani et al., 2024a), disassembly cells involving collaborative robots, or rebalancing tasks. These applications can yield valuable data, enhancing models implemented by embodiments and improving real-world environments.

Embodiments carry significant implications and potential benefits for industrial applications. For real-world industrial settings, the implementation of embodiments allows for the integration of managerial insights and ergonomic expertise into the design process. As industries increasingly move towards automation and human-robot collaboration, ergonomic considerations become crucial. Embodiments, based on a fuzzy knowledge-based expert system, align with the human-centric values of Industry 5.0, representing a shift from the purely technical focus often seen in Industry 4.0 literature. This approach emphasizes the well-being of workers as a priority.

Embodiments also address gaps in the Industry 4.0 framework by integrating ergonomic expertise into the design phase of workplaces, ensuring that worker safety and comfort are considered from the outset. This proactive approach not only optimizes immediate productivity, but also contributes to the long-term sustainability and resilience of the workforce.

Embodiment exemplify this shift towards prioritizing worker well-being and personalized solutions. Embodiments offer a unique feature that allows for the evaluation of cumulative and integrated ergonomic risks across categories of potential workers, including different percentiles of female and male operators (e.g., 5th percentile of female, 50th percentile of female or male, and 95th percentile of male). This capability makes embodiments highly adaptable to specific industry requirements, such as the integration of supportive robots, cobots, and/or specialized equipment designed to mitigate potential risks. By customizing the ergonomic assessments based on the physical characteristics of different worker groups, industries can optimize workplaces for all employees, reducing injury risks and improving overall efficiency.

Unlike existing models, which evaluate the integrated risk of the upper limb or the whole body, an embodiment can provide a comprehensive assessment across all body parts individually in virtual environments. Embodiments represent a pioneering effort in developing an EAT specifically suited for DHM systems. Thus, the ability to evaluate cumulative risk in each body part opens new possibilities for industrial applications, such as assigning appropriate supportive robots to workstations or implementing more effective ergonomic-based job rotation strategies in assembly and disassembly lines. This approach not only enhances worker safety but also contributes to the overall productivity and sustainability of industrial operations.

It is noted that while embodiments are described herein in relation to various EATs, e.g., OCRA, RULA, REBA, and Ergo4All™, embodiments can utilize and/or be varied in accordance with any existing EAT and/or developed EAT.

### Example Advantages

Embodiments provide comprehensive and innovative ergonomic risk assessment models designed to address the challenges and limitations of traditional EATs. By integrating insights from established tools like OCRA, RULA, and REBA, embodiments offer a refined approach for assessing cumulative and integrated ergonomic risks across various body parts. The validation described herein, through real-world and synthesized scenarios, demonstrates the reliability and alignment of embodiments with benchmark EATs, highlighting the potential of embodiments to provide more precise and holistic ergonomic evaluations.

Embodiments contribute significantly to both academic research and industrial practice. Academically, embodiments introduce a robust expert system infrastructure that facilitates the development of fuzzy rules based on ergonomic expertise, paving the way for future studies to refine and optimize ergonomic risk assessments. In industrial settings, the ability of embodiment to assess cumulative risks across diverse worker groups supports the design of safer, more efficient workplaces, aligning with the human-centric values of Industry 5.0. By prioritizing worker well-being, i.e., health, and integrating ergonomic considerations into the early stages of workplace design, embodiment not only enhance productivity, but also promote long-term sustainability and workforce resilience.

Embodiments represents a significant advancement in ergonomic risk assessment, offering a versatile and adaptable tool for both academic exploration and industrial application. By bridging the gap between theoretical research and practical implementation, embodiments improve ergonomic design and assessment and contribute to safer and more sustainable industrial practices.

### Computer Support

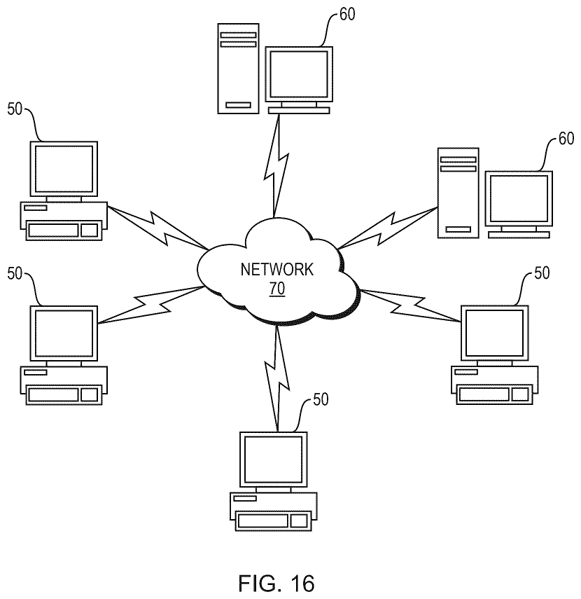

FIG. 16 is a schematic view of a computer network in which embodiments may be implemented. Client computer(s)/devices 50 and server computer(s) 60 provide processing, storage, and input/output (I/O) devices executing application programs and the like. Client computer(s)/device(s) 50 can also be linked through communications network 70 to other computing devices, including other client device(s)/processor(s) 50 and server computer(s) 60. The communications network 70 can be part of a remote access network, a global network (e.g., the Internet), cloud computing servers or service, a worldwide collection of computers, local area or wide area networks, and gateways that currently use respective protocols (e.g., TCP/IP, Bluetooth®, etc.) to communicate with one another. Other electronic device/computer network architectures are also suitable.

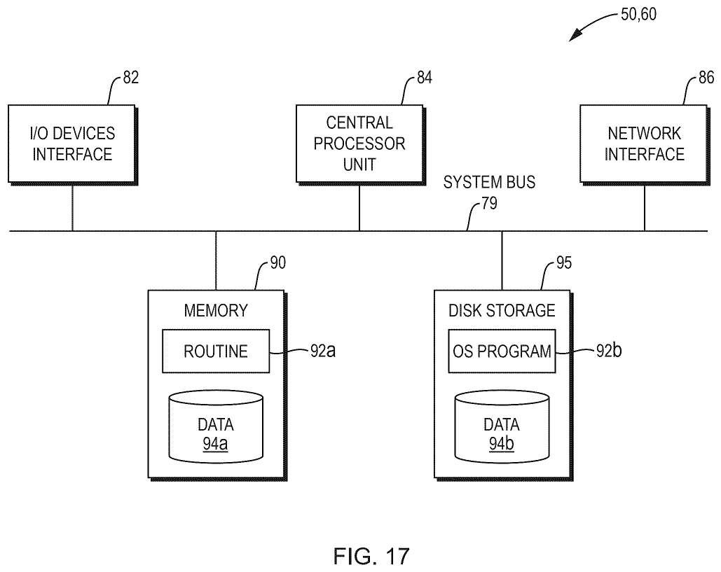

FIG. 17 is a block diagram illustrating an example embodiment of a computer node (e.g., client processor(s)/device(s) 50 or server computer(s) 60) in the computer network 70 of FIG. 16. Each computer node 50, 60 contains system bus 79, where a bus is a set of hardware lines used for data transfer among components of a computer or processing system. The system bus 79 is essentially a shared conduit that connects different elements of a computer system (e.g., processor, disk storage, memory, I/O ports, network ports, etc.) that enables transfer of information between the elements. Attached to the system bus 79 is an I/O devices interface 82 for connecting various input and output devices (e.g., keyboard, mouse, display(s), printer(s), speaker(s), etc.) to the computer node 50, 60. A network interface 86 allows the computer node to connect to various other devices attached to a network (e.g., the network 70 of FIG. 16). A memory 90 provides volatile storage for computer software instructions 92a and data 94a used to implement an embodiment of the present disclosure (e.g., any one or multiple of the methods described herein above, e.g., method 100 or method 700). A disk storage 95 provides non-volatile storage for the computer software instructions 92b and data 94b used to implement an embodiment of the present disclosure. A central processor unit 84 is also attached to the system bus 79 and provides for execution of computer instructions.

In one embodiment, the processor routines 92a-92b and data 94a-94b are a computer program product (generally referenced as 92), including a non-transitory, computer readable medium (e.g., a removable storage medium such as DVD-ROM(s), CD-ROM(s), diskette(s), tape(s), etc.) that provides at least a portion of the software instructions for the disclosed system. The computer program product 92 can be installed by any suitable software installation procedure, as is well known in the art. In another embodiment, at least a portion of the software instructions may also be downloaded over a cable, communication, and/or wireless connection. In other embodiments, the disclosure programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present disclosure routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other networks (such as the network 70 of FIG. 16). In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of the computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium, and the like.

In other embodiments, the program product 92 may be implemented as a so-called Software as a Service (SaaS), or other installation or communication supporting end-users.

Embodiments or aspects thereof may be implemented in the form of hardware including but not limited to hardware circuitry, firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, hardware, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

For example, the foregoing description and details of embodiments in the figures reference Applicant-Assignee (Dassault Systemes Americas Corporation) and Dassault Systemes, tools and platforms, for purposes of illustration and not limitation. Other similar tools and platforms are suitable.

### REFERENCES

Ahmed, S., Irshad, L., Gawand, M. S., & Demirel, H. O. (2021). Integrating human factors early in the design process using digital human modelling and surrogate modelling. Journal of Engineering Design, 32(4), 165-186.
Beuss, F., Sender, J., & Fluegge, W. (2023). Automated simulation-based design of ergonomic workstations using RPA and Human-in-the-Loop decision making. Procedia CIRP, 119, 216-221.
Bourret, Q., Charland, J., Imbeau, D., Brouillette, D., & Djire, J. B. (2021 May). Ergo4All: An Ergonomic Guidance Tool for Non-ergonomist. In Congress of the International Ergonomics Association (pp. 382-390). Cham: Springer International Publishing.
Da Silva, A. G., Mendes Gomes, M. V., & Winkler, I. (2022). Virtual reality and digital human modeling for ergonomic assessment in industrial product development: a patent and literature review. Applied Sciences, 12(3), 1084.
Dahibhate, G., Shinde, R., & Sakle, N. (2023). The use of digital human modeling in ergonomic design and product development. Journal of The Institution of Engineers (India): Series C, 104(5), 1133-1138.
Dassault Systèmes, Ergonomic Workplace Design. URL: https://discover.3ds.com/ergonomic-workplace-design. accessed 12.07.24
De Magistris, G., Micaelli, A., Evrard, P., Andriot, C., Savin, J., Gaudez, C., & Marsot, J. (2013). Dynamic control of DHM for ergonomic assessments. International Journal of Industrial Ergonomics, 43(2), 170-180.
Chaffin, D. B. (2005). Improving digital human modelling for proactive ergonomics in design. Ergonomics, 48(5), 478-491.
Colombini, D., Occhipinti, E., & Álvarez-Casado, E. (2013). The revised OCRA Checklist method. Editorial Factors Humans: Barcelona, Spain.
Emir, T., Varol, T., & Ã-zel, H. B. (2022). Computer-Assisted Ergonomic Analysis of Working Postures Causing Strain. European Journal of Forest Engineering, 8(2), 66-76.
Ghorbani, E., Keivanpour, S., Sekkay, F., & Imbeau, D. (2023a). Ergonomic Assembly Line Balancing Problems Evolution and Future Trends with Insights into Industry 5.0 Paradigm. Bureau de Montreal, Université de Montreal.
Ghorbani, E., Keivanpour, S., Sekkay, F., & Imbeau, D. (2023b). Optimization Framework for Assembly Line Design Problem with Ergonomics Consideration in Fuzzy Environment. In IFIP International Conference on Product Lifecycle Management (pp. 333-343). Cham: Springer Nature Switzerland.
Ghorbani, E., Keivanpour, S., Sekkay, F., & Imbeau, D. (2024a). Fuzzy Ergonomic Expert System for Assembly Line Design Problem. Accessibility, Assistive Technology and Digital Environments, 121(121).
Ghorbani, E., Keivanpour, S., Sekkay, F., & Imbeau, D. (2024b). Fuzzy Fatigue Model for Ergonomic Design of Assembly Lines Under Uncertainty. In Intelligent Systems Conference (pp. 392-407). Cham: Springer Nature Switzerland.
Khamaisi, R. K., Perini, M., Morganti, A., Placci, M., Grandi, F., Peruzzini, M., & Botti, L. (2024). An innovative integrated solution to support digital postural assessment using the TACOs methodology. Computers & Industrial Engineering, 194, 110376.
Kulac, S., & Kiraz, A. (2024). An integrated ergonomic risk assessment framework based on fuzzy logic and IVSF-AHP for optimising ergonomic risks in a mixed-model assembly line. Ergonomics, 1-21.
Lavatelli, I., Schaub, K., & Caragnano, G. (2012). Correlations in between EAWS and OCRA Index concerning the repetitive loads of the upper limbs in automobile manufacturing industries. Work, 41(Supplement 1), 4436-4444.
Manghisi, V. M., Evangelista, A., & Uva, A. E. (2022). A virtual reality approach for assisting sustainable human-centered ergonomic design: the ErgoVR tool. Procedia Computer Science, 200, 1338-1346.
Paudel, P., Kwon, Y. J., Kim, D. H., & Choi, K. H. (2022). Industrial ergonomics risk analysis based on 3d-human pose estimation. Electronics, 11(20), 3403.
Poirson, E., & Delangle, M. (2013 June). Comparative analysis of human modeling tools. In International Digital Human Modeling Symposium.
Tong, Y., & Liu, J. (2021). Review of research and development of supernumerary robotic limbs. IEEE/CAA Journal of Automatica Sinica, 8(5), 929-952.
Yin, M. Y., & Li, J. G. (2023). A systematic review on digital human models in assembly process planning. The International Journal of Advanced Manufacturing Technology, 125(3), 1037-1059.
Ghorbani, E., Keivanpour, S., Sekkay, F., & Imbeau, D. (2024c). Fuzzy expert system for ergonomic assembly line worker assignment and balancing problem under uncertainty. Journal of Industrial and Production Engineering, 42 (3), 274-296. https://doi.org/10.1080/21681015.2024.2389963

1. A computer-implemented method of assessing cumulative ergonomic risk, the method comprising, by a processor:
in memory of the processor, receiving risk data, wherein the risk data includes, for each task of a plurality of tasks performed by an operator, an indication of an ergonomic risk level, of a plurality of ergonomic risk levels, for the operator performing the task;
restructuring the risk data received to determine, across the plurality of tasks, a total time duration for each joint of a plurality of joints of the operator, at each risk level; and
determining the cumulative ergonomic risk based on the total time duration for each joint at each risk level.

2. The computer-implemented method of claim 1, wherein the indication of the ergonomic risk level for each task of the plurality of tasks includes a respective indication of ergonomic risk level for each joint of the plurality of joints of the operator performing the task.

3. The computer-implemented method of claim 1, further comprising:
determining a cumulative ergonomic risk level for a subset of joints of the plurality of joints based on the total time duration for each joint of the subset at each risk level.

4. The computer-implemented method of claim 3, wherein the subset of joints comprises (i) a right shoulder joint, a right elbow joint, and a right wrist joint, (ii) a left shoulder joint, a left elbow joint, and a left wrist joint, or (iii) neck joints and back joints.

5. The computer-implemented method of claim 1, wherein the cumulative ergonomic risk determined includes, for each joint of the plurality of joints, a respective indication of cumulative ergonomic risk across the plurality of tasks.

6. The method of claim 5, wherein each respective indication of cumulative ergonomic risk is a given indication of ergonomic risk level from amongst the plurality of ergonomic risk levels.

7. The method of claim 5, wherein determining the cumulative ergonomic risk comprises:
for each joint of the plurality of joints, determining the respective indication of cumulative ergonomic risk across the plurality of tasks based upon (i) a comparison between the determined total time duration for the joint at a first risk level and a total time duration of the plurality of tasks and (ii) a comparison between the determined total time duration for the joint at a second risk level and the total time duration of the plurality of tasks.

8. The method of claim 7, wherein the first risk level is a high risk level and the second risk level is a medium risk level.

9. The computer-implemented method of claim 1, wherein the plurality of tasks form an operation.

10. The computer-implemented method of claim 1, wherein a first subset of the plurality of tasks form a first operation and a second subset of the plurality of tasks form a second operation and determining the cumulative ergonomic risk based on the total time duration for each joint at each risk level comprises:
identifying a cumulative ergonomic risk of the operator performing the first operation; and
identifying a cumulative ergonomic risk of the operator performing the second operation.

11. The computer-implemented method of claim 1, wherein at least one indication of ergonomic risk level is a function of operator posture and operator exerted force.

12. The computer-implemented method of claim 1, wherein the risk data received comprises data captured by a wearable device on an operator.

13. The method of claim 1, further comprising:
responsive to the cumulative ergonomic risk exceeding a threshold, iteratively (i) determining modified risk levels for the operator performing each task of the plurality of tasks under modified operational conditions, (ii) restructuring the modified risk levels to determine, across the plurality of tasks, a modified total time duration for each joint, at each risk level, and (iii) determining modified cumulative ergonomic risk based on the modified total time duration for each joint at each risk level indicated, until the modified cumulative ergonomic risk is below the threshold.

14. The method of claim 13, further comprising:
modifying a real-world environment in accordance with the modified operational conditions for which the modified cumulative risk is below the threshold.

15. A system for assessing cumulative ergonomic risk, the system comprising:
a processor; and
a memory with computer code instructions stored thereon, the processor and the memory, with the computer code instructions, being configured to cause the system to:
receive risk data, wherein the risk data includes, for each task of a plurality of tasks performed by an operator, an indication of an ergonomic risk level, of a plurality of ergonomic risk levels, for the operator performing the task;
restructure the risk data received to determine, across the plurality of tasks, a total time duration for each joint of a plurality of joints of the operator, at each risk level; and
determine the cumulative ergonomic risk based on the total time duration for each joint at each risk level.

16. The system of claim 15 wherein, the processor and the memory, with the computer code instructions, are further configured to cause the system to:
determine a cumulative ergonomic risk level for a subset of joints of the plurality of joints based on the total time duration for each joint of the subset at each risk level.

17. The system of claim 15, wherein the cumulative ergonomic risk determined includes, for each joint of the plurality of joints, a respective indication of cumulative ergonomic risk across the plurality of tasks.

18. The system of claim 17 where, in determining the cumulative ergonomic risk, the processor and the memory, with the computer code instructions, are further configured to cause the system to:
for each joint of the plurality of joints, determine the respective indication of cumulative ergonomic risk across the plurality of tasks based upon (i) a comparison between the determined total time duration for the joint at a first risk level and a total time duration of the plurality of tasks and (ii) a comparison between the determined total time duration for the joint at a second risk level and the total time duration of the plurality of tasks.

19. The system of claim 15 wherein, the processor and the memory, with the computer code instructions, are further configured to cause the system to:
responsive to the cumulative ergonomic risk exceeding a threshold, iteratively (i) determine modified risk levels for the operator performing each task of the plurality of tasks under modified operational conditions, (ii) restructure the modified risk levels to determine, across the plurality of tasks, a modified total time duration for each joint, at each risk level, and (iii) determine modified cumulative ergonomic risk based on the modified total time duration for each joint at each risk level indicated, until the modified cumulative ergonomic risk is below the threshold.

20. A non-transitory computer program product for assessing cumulative ergonomic risk, the computer program product comprising:
a non-transitory computer readable medium, the non-transitory computer readable medium comprising program instructions which, when executed by a processor, causes the processor to:
receive risk data, wherein the risk data includes, for each task of a plurality of tasks performed by an operator, an indication of an ergonomic risk level, of a plurality of ergonomic risk levels, for the operator performing the task;
restructure the risk data received to determine, across the plurality of tasks, a total time duration for each joint of a plurality of joints of the operator, at each risk level; and
determine the cumulative ergonomic risk based on the total time duration for each joint at each risk level.
